SEQUENCE LISTING (14 sequences)

SEQ ID NO: 1 - protein, 256 aa, respiratory syncytial virus
METYVNKLHEGSTYTAAVQYNVLEKDDDPASLTIWVPMFQSSMPADLLIKELANVNILVKQISTPKGPSLRVMINSRSAVLAQMPSKFTICANVSLDERSKLAYDVTTPCEIKACSLTCLKSKNMLTTVKDLTMKTLNPTHDIIALCEFENIVTSKKVIIPTYLRSISVRNKDLNTLENITTTEFKNAITNAKIIPYSGLLLVITVTDNKGAFKYIKPQSQFIVDLGAYLEKESIYYVTTNWKHTATRFAIKPMED

SEQ ID NO: 2 - protein, 298 aa, respiratory syncytial virus
MSKNKDQRTAKTLERTWDTLNHLLFISSCLYKLNLKSVAQITLSILAMIISTSLIIAAIIFIASANHKVTPTTAIIQDATSQIKNTTPTYLTQNPQLGISPSNPSEITSQITTILASTTPGVKSTLQSTTVKTKNTTTTQTQPSKPTTKQRQNKPPSKPNNDFHFEVFNFVPCSICSNNPTCWAICKRIPNKKPGKKTTTKPTKKPTLKTTKKDPKPQTTKSKEVPTTKPTEEPTINTTKTNIITTLLTSNTTGNPELTSQMETFHSTSSEGNPSPSQVSTTSEYPSQPSSPPNTPRQ

SEQ ID NO: 3 - protein, 574 aa, respiratory syncytial virus
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 4 - protein, 391 aa, respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 5 - DNA, 1725 bp, respiratory syncytial virus
atggagctgctcatcttgaaggctaacgccattaccactatccttacagcggtgacgttctgctttgcatccggtcagaatattaccgaagagttctaccaatctacttgtagcgctgtctcaaaaggctatctgtcggccctccgtacaggatggtacacgagtgttatcaccatcgaattgtccaacattaaggagaataaatgcaacggtactgacgcgaaggtaaagcttatcaaacaggaactggataagtacaagaacgcagtgacagagctccaattgctgatgcagtctaccccccctacgaataaccgcgctaggagagaacttccacgattcatgaactatactctcaataacgccaaaaagaccaacgtcacattgagcaaaaagcgtaagcgcaggtttctgggcttcctcctgggagttggttcagctattgcgtcgggcgtagccgtgagtaaagtccttcacttggagggagaagttaataagatcaagtccgcactcctgtctactaacaaagctgtggtcagcttgtcaaacggtgtatccgtgctgacctcgaaggttcttgacctcaaaaattacatcgataagcaattgctgccgattgtcaacaagcagagttgttctatcagcaatattgagacggtgatcgagttccaacagaaaaacaacagactcctggaaatcacacgtgagttttcagtaaatgccggcgttactacccccgtctccacgtacatgcttacaaactcggaattgctcagtctgattaacgacatgcctatcactaatgatcagaagaagcttatgtctaacaacgtgcaaattgtccgccagcaaagctattccatcatgtcaatcattaaagaggaagtgttggcgtacgtagttcagctcccactgtacggagtcatcgacaccccgtgctggaagcttcatacctcgcccttgtgtacgacaaatactaaagagggttctaacatttgcctcaccaggacggatcgaggctggtattgcgataacgctggaagtgtgagcttcttccctcaagcagaaacatgtaaggtacagtccaatagagttttttgcgacactatgaactcactgacccttccatctgagatcaatttgtgtaacgtcgatatcttcaacccgaagtacgactgcaaaattatgacgtccaagacagatgtgtcgagtagcgtaatcacttcactcggtgccatcgtttcttgctacggcaagaccaaatgtacggcttccaataagaaccgtggaattatcaaaacattctcgaacggttgcgactatgtcagcaataagggcatggacactgtgagtgtaggaaacaccctgtactacgttaacaagcaagaaggtaaatcactgtatgtcaagggcgagcccattatcaatttttacgatcctcttgtgttcccatccgacgagttcgatgcgtctatcagccaggtaaacgaaaagattaaccagtccttggcatttatccgcaaatcggacgagctcctgcacaatgttaacgccggaaagagtacgacaaacattatgatcactaccatcattatcgtcattatcgtgatccttttgtcactcattgctgtaggtctgcttttgtactgtaaagcgaggtctacgcccgttacactcagcaaggatcaactgtccggcatcaataacattgccttctcgaattaa

SEQ ID NO: 6 - DNA, 897 bp, respiratory syncytial virus
atgtccaagaacaaagaccagcgtaccgctaagactctggagcgcacatgggatacgctcaatcacttgcttttcatctctagctgcctgtacaaactcaacttgaagtcagtggcccaaattaccctttcgatcctggcgatgattatcagtacttccctcatcattgcagctatcatttttatcgcctctgcgaatcataaggtcacacccacgaccgcaatcattcaggacgctactagccaaatcaaaaacacaacccctacgtatttgactcagaacccacaactgggtatttcaccgtcgaatcccagtgaaatcacctcccagatcacaactattcttgcctctaccacgcctggcgttaagagcacactccaatcaactaccgtaaagacgaaaaacacaactaccacccagacgcagccatccaagccgacaactaaacaaaggcagaacaagcccccttcgaagccaaataacgatttccacttcgaggtgtttaacttcgtcccgtgtagtatctgctctaataaccccacctgttgggctatttgcaaaagaatccctaacaagaagccaggaaaaaagacgacaactaaacccaccaagaagcctacgttgaaaacaactaagaaggacccgaaaccacaaaccacgaagagcaaagaagttcccacaactaagcctaccgaggaaccgacgatcaatacaactaagaccaacattatcacgacactgctcacttcaaataccactggtaacccagagctgacctcccagatggaaaccttccattcgacgagttctgagggcaaccccagcccttcccaagtatcaacaacttcggaatacccatctcagcccagtagccctccgaataccccacgacaataa

SEQ ID NO: 7 - DNA, 771 bp, respiratory syncytial virus
atggagacctacgtgaacaagctgcacgaaggttccacttatacagctgccgtccagtacaatgttctcgagaaagacgatgaccccgcgtctttgacgatctgggttcctatgttccaaagctcaatgccagcagatcttctgattaaggaactcgctaacgtgaatatcttggtcaaacagatttcgaccccgaagggcccctcgcttcgtgttatgatcaactcccgctctgccgtactggcgcaaatgcctagcaagtttactatctgcgcaaacgtgtcactcgacgagaggtcgaaattggcttacgatgtcacaacgccatgtgaaattaaggcctgcagtctgacctgtcttaagtccaaaaatatgctcactacagttaaggacttgaccatgaaaacgctgaacccgactcatgacatcattgctctctgcgagttcgaaaacatcgtgacctctaagaaggtcatcattcccacatatctgagaagcatctcagtacgaaataaagatcttaacactttggagaacattaccacgacagaatttaagaatgcgatcactaacgccaagatcattccttactccggactcctgttggtgatcaccgttacggacaacaaaggtgcatttaagtacattaaaccacagtcgcaattcatcgtcgatctgggcgcttatcttgagaaggagagtatctactacgtgacaactaattggaagcacaccgccacccgtttcgcgattaaaccgatggaagattaa

SEQ ID NO: 8 - DNA, 1203 bp, respiratory syncytial virus
ggtaccggatccgccaccatggctctgtccaaggtcaagctgaacgacaccctgaacaaggaccagctgctgtcctcctccaagtacaccatccagcgttccaccggtgactccatcgacacccccaactacgacgtgcagaagcacatcaacaagctgtgcggcatgctgctgatcaccgaggacgctaaccacaagttcaccggtctgatcggcatgctgtacgctatgtcccgtctgggtcgtgaggacaccatcaagatcctgcgtgacgctggttaccacgtgaaggctaacggtgtcgacgtgaccacccaccgtcaggacatcaacggcaaggagatgaagttcgaggtcctgaccctggcttccctgaccaccgagatccagatcaacatcgagatcgagtcccgtaagtcctacaagaagatgctgaaggagatgggcgaggtcgcccccgagtaccgtcacgactcccccgactgcggcatgatcatcctgtgcatcgctgctctcgtcatcaccaagctggctgctggtgaccgttccggtctgaccgctgtgatccgtcgtgctaacaacgtgctgaagaacgagatgaagcgctacaagggtctgctgcccaaggacatcgctaacagcttctacgaggtgttcgagaagcacccccacttcatcgacgtgttcgtgcacttcggtatcgctcagtcctccacccgtggtggttcccgtgtggagggcatcttcgctggtctgttcatgaacgcttacggtgctggccaggtcatgctgcgttggggtgtgctggctaagtccgtgaagaacatcatgctgggtcacgcttccgtgcaggctgagatggagcaggtggtggaggtgtacgagtacgctcagaagctgggcggcgaggctggtttctaccacatcctgaacaaccccaaggcttccctgctgtccctgacccagttcccccacttctcctccgtggtgctgggtaacgctgctggtctgggtatcatgggcgagtaccgtggcaccccccgtaaccaggacctgtacgacgctgctaaggcttacgccgagcagctcaaggagaacggcgtcatcaactactccgtgctggacctgaccgctgaggagctggaggctatcaagcaccagctgaaccccaaggacaacgacgtggagctgtaataaaagctt

SEQ ID NO: 9 - protein, 405 aa, Artificial Sequence
  RSV F1-Indo is an engineered protein incorporating sequences from respiratory syncitial virus as well as sequences from influenza virus
MEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMQILSIYSTVASSLALAIMMAGLSLWMCSNGSLQCRICI

SEQ ID NO: 10 - DNA, 1278 bp, Artificial Sequence
  RSV F1-Indo is an engineered gene incorporating sequences from respiratory syncitial virus as well as sequences from influenza virus
tcccaccatcgggcgcggatccgccaccatggagggcgaggtgaacaagatcaagtccgctctgctgtccaccaacaaggctgtggtgtccctgtccaacggtgtctccgtgctgacctccaaggtgctggacctgaagaactacatcgacaagcagctgctgcccatcgtgaacaagcagtcctgctccatctccaacatcgagaccgtgatcgagttccagcagaagaacaaccgtctgctcgagatcacccgtgagttctccgtgaacgctggtgtcaccacccccgtgtccacctacatgctgaccaactccgagctgctgtccctgatcaacgacatgcccatcaccaacgaccaaaagaagctgatgtccaacaacgtgcagatcgtgcgtcagcagtcctactctatcatgtccatcatcaaggaagaagtcctggcttacgtcgtgcagctgcccctgtacggtgtcatcgacaccccctgctggaagctgcacacctcccccctgtgcaccaccaacaccaaggaaggttccaacatctgcctgacccgtaccgaccgtggttggtactgcgacaacgctggttccgtgtccttcttcccccaagctgagacctgcaaggtgcagtccaaccgtgtgttctgcgacaccatgaactccctgaccctgccctccgagatcaacctgtgcaacgtggacatcttcaaccccaagtacgactgcaagatcatgacctctaagaccgacgtgtcctcctccgtgatcacctccctgggtgctatcgtgtcctgctacggcaagaccaagtgcaccgcttccaacaagaaccgcggcatcatcaagaccttctccaacggttgcgactacgtgtccaacaagggcatggacaccgtgtccgtcggtaacaccctgtactacgtcaacaagcaggaaggcaagtctctgtacgtgaagggcgagcccatcatcaacttctacgaccccctggtgttcccctccgacgagttcgacgcttccatcagccaggtcaacgagaagatcaaccagtccctggctttcatccgtaagtccgacgagctgctgcacaacgtcaacgctggcaagtctaccaccaacatcatgcagatcctgtccatctactccaccgtggcttcctccctggctctggctatcatgatggctggtctgtccctgtggatgtgctccaacggctccctgcagtgccgtatctgcatctaataaaagcttgtcgagaagtactagaggatcat

SEQ ID NO: 11 - protein, 447 aa, respiratory syncytial virus
MELLILKANAITTILTAVTFCFASGQNSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 12 - DNA, 1380 bp, respiratory syncytial virus
ccatcgggcgcggatccgccaccatggagctgctgatcctgaaggctaacgctatcaccaccatcctgaccgctgtgaccttctgcttcgcttccggccagaactccaaggtgctgcacctcgagggcgaggtgaacaagatcaagtccgctctgctgtccaccaacaaggctgtggtgtccctgtccaacggtgtctccgtgctgacctccaaggtcctggacctgaagaactacatcgacaagcagctgctgcccatcgtgaacaagcagtcctgctccatctccaacatcgagaccgtgatcgagttccagcagaagaacaaccgtctgctcgagatcacccgtgagttctccgtgaacgctggtgtcaccacccccgtgtccacctacatgctgaccaactccgagctgctgtccctgatcaacgacatgcccatcaccaacgaccagaaaaagctgatgtccaacaacgtgcagatcgtgcgtcagcagtcctactctatcatgtccatcatcaaggaagaggtcctggcttacgtggtgcagctgcccctgtacggtgtcatcgacaccccctgctggaagctgcacacctcccccctgtgcaccaccaacaccaaggaaggttccaacatctgcctgacccgtaccgaccgtggttggtactgcgacaacgctggttccgtgtccttcttcccccaagctgagacctgcaaggtgcagtccaaccgtgtgttctgcgacaccatgaactccctgaccctgccctccgagatcaacctgtgcaacgtggacatcttcaaccccaagtacgactgcaagatcatgacctctaagaccgacgtgtcctcctccgtgatcacctccctgggtgctatcgtgtcctgctacggcaagaccaagtgcaccgcttccaacaagaaccgcggcatcatcaagaccttctccaacggttgcgactacgtgtccaacaagggcatggacaccgtgtccgtcggtaacaccctgtactacgtcaacaagcaggaaggcaagtctctgtacgtgaagggcgagcccatcatcaacttctacgaccccctggtgttcccctccgacgagttcgacgcttccatcagccaggtcaacgagaagatcaaccagtccctggctttcatccgtaagtccgacgagctgctgcacaacgtcaacgctggcaagtctaccaccaacatcatgatcaccactatcatcatcgtgatcatcgtcatcctgctgtctctcatcgctgtgggtctgctgctgtactgcaaggctcgttccacccctgtgaccctgtccaaggaccagctgtccggtatcaacaacatcgctttctccaactaatgaaagcttgtcg

SEQ ID NO: 13 - DNA, 759 bp, Influenza virus
atgagtcttctaaccgaggtcgaaacgtacgttctctctatcatcccgtcaggccccctcaaagccgagatcgcgcagaaacttgaagatgtctttgcaggaaagaacaccgatctcgaggctctcatggagtggctgaagacaagaccaatcctgtcacctctgactaaagggattttgggatttgtattcacgctcaccgtgcccagtgagcgaggactgcagcgtagacgctttgtccagaatgccctaaatggaaatggagatccaaataatatggatagggcagttaagctatataagaagctgaaaagagaaataacattccatggggctaaagaggtttcactcagctactcaaccggtgcacttgccagttgcatgggtctcatatacaacaggatgggaacggtgactacggaagtggcttttggcctagtgtgtgccacttgtgagcagattgcagattcacagcatcggtctcacaggcagatggcaactatcaccaacccactaatcaggcatgaaaacagaatggtgctggccagcactacagctaaggctatggagcagatggcgggatcaagtgagcaggcagcggaagccatggaggtcgctaatcaggctaggcagatggtgcaggcaatgaggacaattggaactcatcctaactctagtgctggtctgagagataatcttcttgaaaatttgcaggcctaccagaaacgaatgggagtgcagatgcagcgattcaagtga

SEQ ID NO: 14 - DNA, 821 bp, Influenza virus
ttcataccgtcccaccatcgggcgcggatccgccaccatgtccctgctgaccgaggtggagacctacgtgctgtccatcgtgccctccggtcctctgaaggctgagatcgctcagaggctcgaggacgtgttcgctggcaagaacaccgacctcgaggctctgatggagtggctcaagacccgtcccatcctgtcccccctgaccaagggtatcctgggtttcgtgttcaccctgaccgtgccttccgagcgtggtctgcagcgtcgtcgtttcgtgcagaacgctctgaacggtaacggtgaccccaacaacatggacaaggccgtgaagctgtaccgtaagctgaagcgtgagatcaccttccacggtgctaaggagatcgctctgtcctactccgctggtgctctggcttcctgcatgggcctgatctacaaccgtatgggtgctgtgaccaccgaggtggccttcggtctggtctgcgctacctgcgagcagatcgctgactcccagcaccgttcccaccgtcagatggtggctaccaccaaccccctgatccgtcacgagaaccgcatggtgctggcttccaccaccgctaaggctatggagcagatggctggttcctccgagcaggctgctgaggccatggagatcgcttcccaggctcgccagatggtgcaggctatgcgtgctatcggcacccacccctcctcctccaccggtctgcgtgacgacctgctcgagaacctgcagacctaccaaaagcgtatgggtgtgcagatgcagcgcttcaagtaataaaagcttgtcgagaagtactaga

(19) United States
(12) Patent Application Publication
(10) Pub. No.: US 2008/0233150 A1
(43) Pub. Date: Sep. 25, 2008

(54) RESPIRATORY SYNCYTIAL VIRUS-VIRUS LIKE PARTICLE (VLPS)

(76) Inventors: Gale SMITH, Rockville, MD (US); Peter Pushko, Rockville, MD (US); Mike Massare, Rockville, MD (US); Yingyun Wu, Rockville, MD (US); Kutub Mahmood, Rockville, MD (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 11/941,768

(22) Filed: Nov. 16, 2007

Related U.S. Application Data
(60) Provisional application No. 60/859,290, filed on Nov. 16, 2006.
(60) Provisional application No. 60/901,652, filed on Feb. 16, 2007.

(51) Int. Cl. A61K 39/155; A61P 31/12; C07K 16/00

(52) U.S. Cl. 424/211.1; 530/387.3

(57) ABSTRACT

The present invention discloses and claims virus like particles (VLPs) that express and/or contains RSV proteins. The invention includes vector constructs comprising said proteins, cells comprising said constructs, formulations and vaccines comprising VLPs of the inventions. The invention also includes methods of making and administrating VLPs to vertebrates, including methods of inducing immunity to infections, including RSV.

This Application claims priority to provisional applications 60/859,290, filed Nov. 16, 2006, and 60/901,652, filed Feb. 16, 2007, which are herein incorporated by reference in their entireties for all proposes.

### BACKGROUND OF THE INVENTION

Respiratory syncytial virus (RSV) is a member of the genus Pneumovirus of the family Paramyxoviridae. This virus has a genome comprised of a single strand negative-sense RNA, which is tightly associated with viral protein to form the nucleocapsid. The viral envelope is composed of a plasma membrane derived lipid bilayer that contains virally encoded structural proteins. A viral polymerase is packaged with the virion and transcribes genomic RNA into mRNA. The RSV genome encodes three transmembrane structural proteins, (F, G, and SH), two matrix proteins (M and M2), three nucleocapsid proteins (N, P and L, and two nonstructural proteins (NS1 and NS1) (Collins et al. (1996) Respiratory syncytial virus, pp. 1313-1351, In B. N. Fields (ed.), Fields virology. Raven Press, New York, N.Y.).

Human respiratory syncytial virus (RSV) is the leading cause of severe lower respiratory tract disease in infants and young children and is responsible for considerable morbidity and mortality in humans. RSV is also recognized as an important agent of disease in immuno-compromised adults and in the elderly. Due to incomplete resistance to RSV in the infected host after a natural infection, RSV may infect multiple times during childhood and adult life.

As stated above, the RSV genome encodes 10 proteins: NS1, NS2, N, P, M, SH, G, F, M2, and L (Collins et al. (1994) J. Virol., 49, 572-578). The M protein is expressed as a peripheral membrane protein, whereas the F and G proteins are expressed as structural membrane proteins and are involved in virus attachment and viral entry into cells. The F and G proteins are the major antigens that elicit neutralizing antibodies in vivo (as reviewed in McIntosh and Chanock, 1990, Respiratory Syncytial Virus, In In Virology, 2nd ed, D. M. Knipe et al., (ed.). Raven Press New York, N.Y.). Antigenic dimorphism between the subgroups of RSV A and B is mainly linked to the G protein, whereas the F protein is more closely related between the subgroups.

Despite decades of research, no safe and effective RSV vaccine has been developed for the prevention of severe morbidity and mortality associated with RSV infection. A formalin-inactivated virus vaccine has failed to provide protection against RSV infection and it exacerbated symptoms during subsequent infection by the wild-type virus in vaccinated infants (Kapikian et al., 1969, Am. J. Epidemiol. 89:405-21; Chin et al., 1969, Am. J. Epidemiol. 89:449-63). Efforts since have focused on developing live attenuated temperature-sensitive mutants by chemical mutagenesis or cold passage of the wild-type RSV (Gharpure et al. (1969) J. Virol., 3, 414-21; Crowe et al. (1994) Vaccine, 12, 691-9). However, earlier trials yielded discouraging results with these live attenuated temperature sensitive mutants. Virus candidates were either underattenuated or overattenuated (Kim et al. (1973) Pediatrics, 52, 56-63; Wright et al (1976) J. Pediatrics, 88, 931-936) and some of the vaccine candidates were genetically unstable which resulted in the loss of the attenuated phenotype (Hodes et al. (1974) Proc. Soc. Exp. Biol. Med., 145, 1158-1164).

Virus-like particles (VLPs) closely resemble mature virions, but they do not contain viral genomic material (i.e., viral genomic RNA). Therefore, VLPs are non-replicative in nature, which make them safe for administration in the form of an immunogenic composition (e.g., vaccine). In addition, VLPs can express structural proteins on the surface of the VLP, which is the most physiological configuration. Moreover, since VLPs resemble intact virions and are multivalent particulate structures, VLPs may be more effective in inducing neutralizing antibodies to the structural protein than soluble envelope antigens. Furthermore, VLPs can be administered repeatedly to vaccinated hosts, unlike many recombinant vaccine approaches. As described herein, the inventors have invented a RSV VLP that can potentially induce an anti-RSV immune response when administered to a vertebrate.

### SUMMARY OF THE INVENTION

The present invention comprises a VLP comprising respiratory syncytial virus (RSV) protein. In one embodiment, said VLP further comprise a RSV F protein. In another embodiment, said VLP comprises a RSV M protein. In another embodiment, said VLP comprises a RSV N protein. In another embodiment, said VLP further comprises RSV G protein. In another embodiment, said G protein is from RSV group A. In another embodiment, said G protein is from RSV group B. In another embodiment, said VLP is expressed in a eukaryotic cell under conditions that permit the formation of VLPs.

The present invention also comprises a VLP comprising a chimeric F protein from RSV and optionally M1 protein from an influenza virus, wherein said chimeric F protein is a fused to the transmembrane domain and cytoplasmic tail of influenza HA protein. In one embodiment, said F protein transmembrane and cytoplasmic domains are replaced with influenza HA protein transmembrane and cytoplasmic domains.

The present invention also comprises a method of producing VLPs, comprising transfecting vectors encoding at least one RSV F protein into a suitable host cell and expressing said RSV virus protein under conditions that allow VLP formation. In one embodiment, said method comprises producing VLPs which comprise a M protein from RSV. In another embodiment, said method comprises producing VLPs which comprise a RSV F protein, wherein said F protein is a chimeric F protein and wherein said chimeric F protein is a fused to the transmembrane domain and cytoplasmic tail of influenza HA protein.

The present invention also comprises an antigenic formulation comprising VLPs which comprise at least one RSV protein. In one embodiment, said antigenic formulation comprises VLPs, wherein said VLPs comprise a RSV F protein. In another embodiment, said antigenic formulation comprises VLPs, wherein said VLPs further comprise a RSV M protein and/or RSV N. In another embodiment, said antigenic formulation further comprises a RSV G protein.

The present invention also comprises an antigenic formulation, comprising VLPs which comprise a chimeric F protein from a RSV and optionally M1 protein derived from an influenza virus, wherein said chimeric F protein is a fused to the transmembrane domain and cytoplasmic tail of influenza HA protein. In another embodiment, said antigenic formulation comprises an adjuvant. In another embodiment, said adjuvant are Novasomes. In another embodiment, said antigenic formulation is suitable for human administration. In another embodiment, said antigenic formulation are blended together to create a multivalent formulation.

The present invention also comprises a vaccine comprising VLPs which comprise at least one RSV protein. In another embodiment, said vaccine comprises VLPs comprising a RSV F protein. In another embodiment, said vaccine comprises VLPs comprising a RSV M protein. In another embodiment, said VLP comprises a RSV N protein. In another embodiment, said vaccine comprises VLPs further comprising a RSV G protein. In another embodiment, said vaccine comprises different antigenic RSV VLPs are blended together to create a multivalent formulation.

The present invention also comprises a vaccine comprising VLPs which comprise a chimeric F protein from a RSV and optionally M1 protein from an influenza virus, wherein said chimeric F protein is a fused to the transmembrane domain and cytoplasmic tail of an influenza protein. In another embodiment, said influenza protein is HA and/or NA.

The present invention also comprises a method of vaccinating a mammal against RSV comprising administering to said mammal a protection-inducing amount of VLPs comprising at least one RSV protein. In one embodiment, said VLPs comprise RSV F and/or M proteins. In another embodiment, said VLPs further comprise RSV F, RSV N and RSV M proteins. In another embodiment, said VLPs comprising a chimeric F protein from RSV and optionally M1 protein derived from an influenza virus, wherein said chimeric F protein is a fused to the transmembrane domain and cytoplasmic tail of influenza HA protein.

The present invention also comprises a method of inducing immunity to RSV infection or at least one symptom thereof in a subject, comprising administering at least one effective dose of RSV VLPs. In one embodiment, said method comprises VLPs comprising a RSV F protein. In another embodiment, said method comprises, VLPs comprising a RSV M protein. In another embodiment, said VLP comprises a RSV N protein. In another embodiment, said method comprises VLPs further comprising a RSV G protein. In another embodiment, said method comprises VLPs comprising a chimeric F protein from a RSV and optionally M1 protein derived from an influenza virus, wherein said chimeric F protein is a fused to the transmembrane domain and cytoplasmic tail of influenza HA protein.

The present invention also comprises a chimeric VLP comprising a viral M from RSV and at least one protein from an infectious agent. In one embodiment, said protein from an infectious agent is a viral protein. In another embodiment, said protein from an infectious agent is an envelope associated protein. In another embodiment, said protein from an infectious agent is expressed on the surface of the VLP. In another embodiment, said protein from another infectious agent is fused to a RSV protein. In another embodiment, said VLPs comprise more than one protein from an infectious agent.

### BRIEF DESCRIPTION OF THE FIGURES

FIG. 1 represents chimeric RSV F proteins comprising influenza HA regions.

FIG. 2 represents RSV protein constructs in a baculovirus genome.

FIG. 3 represents the cloning strategy and maps for RSV G, RSV F, RSV M, RSV N, RSV FM and RSV GFM genes into Bacmid vectors.

FIG. 4 represents SDS stained gel and western blot of recombinant baculovirus expression in insect cells.

FIG. 5 is an electron micrograph of RSV VLPs with ammonium molybdate staining. The shows rod shaped particles are baculovirus and round particles are RSV-VLPs.

FIG. 6 is a western blot of VLPs isolated through a 30% sucrose cushion.

FIG. 7 is a western blot of VLPs isolated through a 30% sucrose cushion.

### DETAILED DESCRIPTION

As used herein the term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein an “effective dose” generally refers to that amount of VLPs of the invention sufficient to induce immunity, to prevent and/or ameliorate an infection or to reduce at least one symptom of an infection and/or to enhance the efficacy of another dose of a VLP. An effective dose may refer to the amount of VLPs sufficient to delay or minimize the onset of an infection. An effective dose may also refer to the amount of VLPs that provides a therapeutic benefit in the treatment or management of an infection. Further, an effective dose is the amount with respect to VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As used herein, the term “effective amount” refers to an amount of VLPs necessary or sufficient to realize a desired biologic effect. An effective amount of the composition would be the amount that achieves a selected result, and such an amount could be determined as a matter of routine experimentation by a person skilled in the art. For example, an effective amount for preventing, treating and/or ameliorating an infection could be that amount necessary to cause activation of the immune system, resulting in the development of an antigen specific immune response upon exposure to VLPs of the invention. The term is also synonymous with “sufficient amount.”

As used herein, the term “multivalent” refers to VLPs which have multiple antigenic proteins against multiple types or strains of infectious agents.

As used herein the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as VLPs of the invention, or can be administered separately. Either the protein or an expression vector encoding the protein can be administered to produce an immunostimulatory effect.

As used herein the term “protective immune response” or “protective response” refers to an immune response mediated by antibodies against an infectious agent, which is exhibited by a vertebrate (e.g., a human), that prevents or ameliorates an infection or reduces at least one symptom thereof. VLPs of the invention can stimulate the production of antibodies that, for example, neutralize infectious agents, blocks infectious agents from entering cells, blocks replication of said infectious agents, and/or protect host cells from infection and destruction. The term can also refer to an immune response that is mediated by T-lymphocytes and/or other white blood cells against an infectious agent, exhibited by a vertebrate (e.g., a human), that prevents or ameliorates RSV infection or reduces at least one symptom thereof.

As use herein, the term “infectious agent” refers to microorganisms that cause an infection in a vertebrate. Usually, the organisms are viruses, bacteria, parasites and/or fungi.

As use herein, the term “antigenic formulation” or “antigenic composition” refers to a preparation which, when administered to a vertebrate, e.g. a mammal, will induce an immune response.

As used herein, the term “vaccine” refers to a formulation which contains VLPs of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As use herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered. In particular, infants and young children are appropriate subjects or patients for a RSV vaccine.

As used herein, the term “virus-like particle” (VLP) refers to a structure that in at least one attribute resembles a virus but which has not been demonstrated to be infectious. Virus-like particles in accordance with the invention do not carry genetic information encoding for the proteins of the virus-like particles. In general, virus-like particles lack a viral genome and, therefore, are noninfectious. In addition, virus-like particles can often be produced in large quantities by heterologous expression and can be easily purified.

A used herein, the term “chimeric VLP” refers to VLPs that contain proteins, or portions thereof, from at least two different infectious agents (heterologous proteins). Usually, one of the proteins is derived from a virus that can drive the formation of VLPs from host cells. Examples, for illustrative purposes, are RSV M and/or influenza M1 protein. The terms RSV VLPs and chimeric VLPs can be used interchangeably where appropriate.

As used herein, the terms “RSV Matrix” or “RSV M” protein refer to a RSV protein that, when expressed in a host cell, induces formation of VLPs. An example of a RSV M protein is represented by SEQ ID No. 1. The term also comprises any variants, derivatives and/or fragments of RSV M that, when expressed in a host cell, induces formation of VLPs. The term also encompasses nucleotide sequences which encode for RSV M and/or any variants, derivatives and/or fragments thereof that when transfected (or infected) into a host cell will express RSV M protein and induce formation of VLPs.

Currently, the only approved approach to prophylaxis of RSV disease is passive immunization. Initial evidence suggesting a protective role for IgG was obtained from observations involving maternal antibody in ferrets (Prince, G. A., Ph.D. diss., University of California, Los Angeles, 1975) and humans (Lambrecht et al., (1976) J. Infect. Dis. 134, 211-217; and Glezen et al. (1981) J. Pediatr. 98, 708-715). Hemming et al. (Morell et al., eds., 1986, Clinical Use of Intravenous Immunoglobulins, Academic Press, London at pages 285-294) recognized the possible utility of RSV antibody in treatment or prevention of RSV infection during studies involving the pharmacokinetics of an intravenous immune globulin (IVIG) in newborns suspected of having neonatal sepsis. They noted that one infant, whose respiratory secretions yielded RSV, recovered rapidly after IVIG infusion. Subsequent analysis of the IVIG lot revealed an unusually high titer of RSV neutralizing antibody. This same group of investigators then examined the ability of hyperimmune serum or immune globulin, enriched for RSV neutralizing antibody, to protect cotton rats and primates against RSV infection (Prince et al. (1985) Virus Res. 3, 193-206; Prince et al. (1990) J. Virol. 64, 3091-3092. Results of these studies suggested that RSV neutralizing antibody given prophylactically inhibited respiratory tract replication of RSV in cotton rats. When given therapeutically, RSV antibody reduced pulmonary viral replication both in cotton rats and in a nonhuman primate model. Furthermore, passive infusion of immune serum or immune globulin did not produce enhanced pulmonary pathology in cotton rats subsequently challenged with RSV.

Two proteins, F and G, on the surface of RSV have been shown to be targets of neutralizing antibodies (Sullender, W. (2000) Clinical Microbiology Review 13, 1-15). These two proteins are also primarily responsible for viral recognition and entry into target cells; G protein binds to a specific cellular receptor and the F protein promotes fusion of the virus with the cell. The F protein is also expressed on the surface of infected cells and is responsible for subsequent fusion with other cells leading to syncytia formation. Thus, antibodies to the F protein can neutralize virus or block entry of the virus into the cell or prevent syncytia formation. Although antigenic and structural differences between A and B subtypes have been described for both the G and F proteins, the more significant antigenic differences reside on the G protein, where amino acid sequences are only 53% homologous and antigenic relatedness is 5% (Walsh et al. (1987) J. Infect. Dis. 155, 1198-1204; and Johnson et al. (1987) Proc. Natl. Acad. Sci. USA 84, 5625-5629). Conversely, antibodies raised to the F protein show a high degree of cross-reactivity among subtype A and B viruses.

The RSV F protein directs penetration of RSV by fusion between the virion's envelope protein and the host cell plasma membrane. Later in infection, the F protein expressed on the cell surface can mediate fusion with neighboring cells to form syncytia. The F protein is a type I transmembrane surface protein that has a N-terminal cleaved signal peptide and a membrane anchor near the C-terminus. RSV F is synthesized as an inactive F0 precursor that assembles into a homotrimer and is activated by cleavage in the trans-Golgi complex by a cellular endoprotease to yield two disulfide-linked subunits. The N-terminus of the F1 subunit that is created by cleavage contains a hydrophobic domain (the fusion peptide) that inserts directly into the target membrane to initiate fusion. The F1 subunit also contains heptad repeats that associate during fusion, driving a conformational shift that brings the viral and cellular membranes into close proximity (Collins and Crowe, 2007, Fields Virology, 5^{th} ed., D. M Kipe et al., Lipincott, Williams and Wilkons, p. 1604). SEQ ID NO 3 depicts a representative RSV F protein. Encompassed in this invention are RSV F proteins that are about 80%, about 85%, about 90%, about 95%, about 96%, about 97%, about 98% or about 99% identical to SEQ ID NO 3, and all fragments and variants (including chimeric proteins) thereof.

RSV M protein is a nonglycosylated internal virion protein that accumulates in the plasma membrane that interacts with RSV F protein and other factors during virus morphogenesis (Id., p. 1608). SEQ ID NO 1 depicts a representative RSV M protein. Encompassed in this invention are RSV M proteins that are about 80%, about 85%, about 90%, about 95%, about 96%, about 97%, about 98% or about 99% identical to SEQ ID NO 1, and all fragments and variants (including chimeric proteins) thereof.

RSV G protein is a type II transmembrane glycoprotein with a single hydrophobic region near the N-terminal end that serves as both an uncleaved signal peptide and a membrane anchor, leaving the C-terminal two-thirds of the molecule oriented externally. RSV G is also expressed as a secreted protein that arises from translational initiation at the second AUG in the ORF (at about amino acid 48), which lies within the signal/anchor. Most of the ectodomain of RSV G is highly divergent between RSV strains (Id., p. 1607). SEQ ID NO 2 depicts a representative RSV G protein. Encompassed in this invention are RSV G proteins that are about 80%, about 85%, about 90%, about 95%, about 96%, about 97%, about 98% or about 99% identical to SEQ ID NO 2, and all fragments and variants (including chimeric proteins) thereof.

RSV N protein binds tightly to both genomic RNA and the replicative intermediate antigenomic RNA to form RNAse resistant nucleocapsid. RSV N may be also required for efficient formation of RSV VLPs. SEQ ID NO 4 depicts a representative RSV 4 protein. Encompassed in this invention are RSV N proteins that are about 80%, about 85%, about 90%, about 95%, about 96%, about 97%, about 98% or about 99% identical to SEQ ID NO 4, and all fragments and variants (including chimeric proteins) thereof.

Since RSV infection can be prevented by providing neutralizing antibodies to a vertebrate, a vaccine comprising RSV F and/or G proteins may induce, when administered to a vertebrate, neutralizing antibodies in vivo. Thus, the invention encompasses RSV VLPs that can be formulated into vaccines or antigenic formulations for protecting vertebrates (e.g. humans) against RSV infection or at least one symptom thereof. The present invention also relates to RSV VLPs and vectors comprising wild-type and mutated RSV genes or a combination thereof derived from different strains of RSV virus, which when transfected into host cells, will produce virus like particles (VLPs) comprising RSV proteins.

In some embodiments, RSV virus-like particles may include at least a viral core protein (e.g. RSV M and/or RSV N proteins) and at least one viral surface envelope protein (e.g. RSV F and/or G proteins). As shown below, the inventors have discovered that expressing RSV M protein in cells induces VLP formation. Thus, one embodiment of the invention comprises RSV VLPs wherein said VLPs are formed from the expression of RSV M protein. In another embodiment, VLPs of the invention comprise at least one viral surface envelope RSV protein incorporated into the VLP. In another embodiment, said envelope RSV protein comprises RSV F protein. The RSV F protein can be from the same or different RSV strain than that of the RSV M protein. In another embodiment, said VLPs further comprises RSV G protein. In another embodiment, the G protein is from RSV group A. In another embodiment, the G protein is from RSV group B. In another embodiment, the RSV F and M proteins are derived from RSV group B and/or group A. In another embodiment, VLPs of the invention comprise RSV N and/or P protein. In another embodiment, the viral core protein and the viral surface envelope protein are from the same virus. The invention also comprises combination of different RSV F, M and/or G proteins from different strains. In addition, said VLPs can include one or more additional molecules for the enhancement of an immune response.

In another embodiment of the invention, said RSV VLPs can carry agents such as nucleic acids, siRNA, microRNA, chemotherapeutic agents, imaging agents, and/or other agents that need to be delivered to a patient.

Chimeric VLPs are VLPs having at least two proteins in said VLPs, wherein one protein can drive VLP formation (e.g. RSV M) and the other protein is from a heterologous infectious agent or from more than one strain, group, subtype etc. of the same agent. Infectious agent proteins may have antigenic variations of the same protein or can be a protein from an unrelated agent. Thus, in one embodiment, chimeric VLPs of the invention comprise RSV F proteins from different RSV strains. For example, chimeric VLPs can comprise F protein from RSV group A and RSV group B. In another embodiment, chimeric VLPs can comprise G protein from RSV group A and RSV group B. In another embodiment, chimeric VLPs can comprise RSV F protein from group A and RSV M protein from group B, or vice a versa. In another embodiment, a chimeric VLPs of the invention can comprise HA and/or NA from influenza virus and F and/or G proteins from RSV.

VLPs of the invention are useful for preparing vaccines and immunogenic compositions. One important feature of VLPs is the ability to express surface proteins so that the immune system of a vertebrate induces an immune response against said protein. However, not all proteins can be expressed on the surface of VLPs. There may be many reasons why certain proteins are not expressed, or be poorly expressed, on the surface of F VLPs. One reason is that said protein is not directed to the membrane of a host cell or that said protein does not have a transmembrane domain. Sequences near the carboxyl terminus of influenza hemagglutinin may be important for incorporation of HA into the lipid bilayer of the mature influenza enveloped nucleocapsids and for the assembly of HA trimer interaction with the influenza core protein M1 (Ali, et al., (2000) J. Virol. 74, 8709-19).

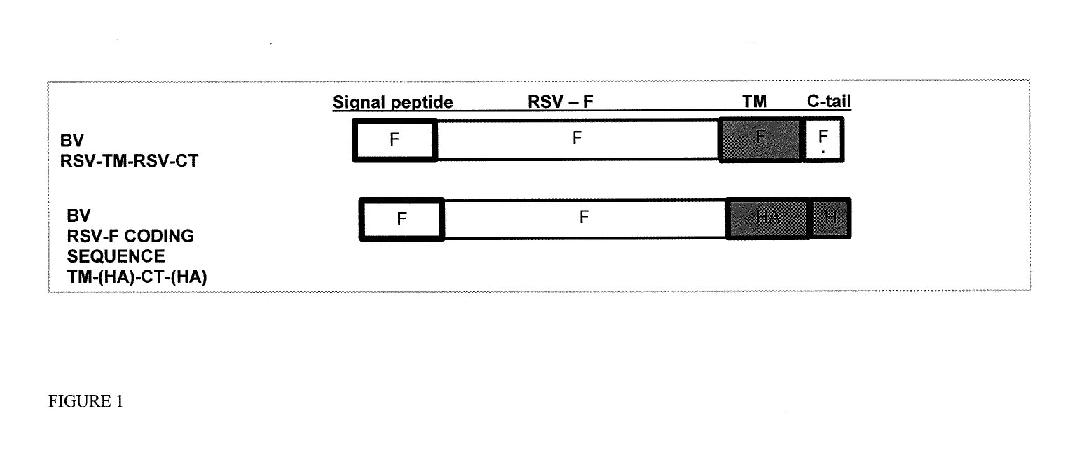

Thus, one embodiment of the invention comprises VLPs comprising a chimeric F protein from RSV and optionally M1 protein from an influenza virus, wherein said chimeric F protein is a fused to the transmembrane domain and cytoplasmic tail of influenza HA protein. In another embodiment, the transmembrane domain and cytoplasmic tail of influenza HA protein are fused to the F protein. In another embodiment, the F protein transmembrane and cytoplasmic domains are removed and replaced with influenza HA protein transmembrane and cytoplasmic domains. In another embodiment, said influenza M1 is from A/Indonesia (H5N1) or A/Fujian (H3N2). In another embodiment, said transmembrane domain and cytoplasmic tail of HA fused to a RSV protein is from A/Indonesia (H5N1). In another embodiment, said VLPs comprising chimeric RSV F protein, and optionally influenza M1 protein, also comprise RSV G protein from RSV group A and/or group B. In another embodiment, said G protein (group A and/or B) is a fused to the transmembrane domain and cytoplasmic tail of influenza HA protein. In another embodiment, said chimeric VLPs comprise a chimeric F protein, NA and/or HA and optionally M1 protein from influenza virus. Examples of the above constructs are illustrated in FIG. 1. These constructs and VLPs take advantage of the efficient system utilized by influenza virus to make virus particles. See co-pending U.S. application 60/817,402, herein incorporated by reference on its entirety for all purposes.

Another embodiment of the invention comprises VLPs comprising a RSV-influenza chimeric protein without having a core (or M) protein. In one embodiment, the said VLP comprise a transmembrane domain and cytoplasmic tail of influenza HA protein fused to the RSV F protein. In another embodiment, said transmembrane domain and cytoplasmic tail of HA is fused to a RSV protein is from A/Indonesia (H5N1).

Other chimeric VLPs of the invention comprise VLPs comprising a RSV M protein and at least one protein from another infectious agent. In one embodiment, said protein from another infectious agent is a viral protein. In another embodiment, said protein from an infectious agent is an envelope-associated protein. In another embodiment, said protein from another infectious agent is expressed on the surface of VLPs. In another embodiment, said protein from an infectious agent comprises an epitope that will generate a protective immune response in a vertebrate. In another embodiment, said protein from another infectious agent is fused to a RSV protein. In another embodiment, only a portion of a protein from another infectious agent is fused to a RSV protein. In another embodiment, only a portion of a protein from another infectious agent is fused to a portion of a RSV protein. In another embodiment, said portion of the protein from another infectious agent fused to RSV protein is expressed on the surface of VLPs. In other embodiment, said RSV protein, or portion thereof, is derived from RSV F, G, N and/or P.

It has been shown that interactions between some viral matrix proteins and envelope proteins may be important for incorporating native envelope proteins into VLPs (Pantua et al. (2006) J. Virol., 80, 11062-11073). Thus, it may be necessary for heterologous proteins to associate with RSV M to make a chimeric RSV VLP. Some heterologous proteins may associate with RSV naturally. However, some heterologous proteins (and even some native RSV proteins) may need to be engineered to make them associate with RSV M (the term associate with RSV M implies both direct association and indirect association). Thus, in one embodiment, said protein from another infectious agent can associated with RSV M protein. In other embodiment said RSV protein, or portion thereof, is fused to a protein from another infectious agent that associates with the RSV M protein.

Another approach to make chimeric VLPs comprising heterologous proteins and RSV M is to engineer chimeric molecules with RSV proteins that can associate with RSV M fused with heterologous proteins (or native RSV proteins). For example, the external domains of proteins from infective agents such as VZV, Dengue, influenza and/or other proteins can be used to generate chimeric molecules by fusing said proteins to RSV proteins that associates with RSV M. Studies have indicated that RSV G and N interact with RSV M. Thus, one embodiment the invention comprises a VLP comprising a chimeric molecule comprising the transmembrane domain and/or cytoplasmic tail of RSV G and/or chimeric RSV N fused to heterologous proteins. In another embodiment, the transmembrane domain and/or cytoplasmic tail of the G protein extends from the N-terminus to the approximately 0, 1, 2, 3 4, 5, 6, 7, 8, 9, 10, 11, 12, 13, 14, 15, 16, 17, 18, 19, 20 to about 50 amino acids past the transmembrane domain and is fused to a protein from another infectious agent. In another embodiment, said portion of the G protein that comprises the cytoplasmic and the transmembrane domain is fused to a portion of the protein from another infectious agent. In another embodiment, said portion of the protein from another infectious agent is HA and/or NA from influenza virus (all serotypes, including avian and human strains). In other embodiment, said HA and/or NA do not comprise their natural cytoplasmic and/or transmembrane domain. In this construct, the cytoplasmic and/or transmembrane domain of HA and/or NA is replaced with the transmembrane and/or cytoplasmic domains of RSV G. In another embodiment, said VLP comprises RSV N and/or P protein. In another embodiment, said protein from an infectious agent is fused to the RSV N protein. In another embodiment, said protein from an infectious agent is HA and/or NA from influenza virus (all serotypes, including avian and human strains). In another embodiment, said protein from an infectious agent is fused to the RSV M protein.

In one embodiment of the invention, the chimeric genes, which may be codon optimized, are synthesized and cloned through a series of steps into a bacmid construct followed by rescue of recombinant baculovirus by plaque isolation and expression analyses. The VLPs for each of these targets can then be rescued by co-infection with the use of two recombinant baculoviruses (1) expressing the RSV M, and (2) expressing the chimeric protein from an infectious agent (e.g. VZV, RSV, Dengue, influenza) with cytoplasmic and transmembrane domain of RSV G. In another embodiment, the VLPs for each of these targets can then be rescued by infection with the use of a recombinant baculovirus expressing the RSV M, and the chimeric protein from an infectious agent (e.g. VZV, RSV, Dengue, influenza) with cytoplasmic and transmembrane domain of RSV G.

Infectious agents can be viruses, bacteria and/or parasites. A protein that may be expressed on the surface of RSV VLPs can be derived from viruses, bacteria and/or parasites. The proteins derived from viruses, bacteria and/or parasites can induce an immune response (cellular and/or humoral) in a vertebrate that which will prevent, treat, manage and/or ameliorate an infectious disease in said vertebrate.

Non-limiting examples of viruses from which said infectious agent proteins can be derived from are the following: influenza (A and B, e.g. HA and/or NA), coronavirus (e.g. SARS), hepatitis viruses A, B, C, D & E3, human immunodeficiency virus (HIV), herpes viruses 1, 2, 6 & 7, cytomegalovirus, varicella zoster, papilloma virus, Epstein Barr virus, parainfluenza viruses, adenoviruses, bunya viruses (e.g. hanta virus), coxsakie viruses, picoma viruses, rotaviruses, rhinoviruses, rubella virus, mumps virus, measles virus, Rubella virus, polio virus (multiple types), adeno virus (multiple types), parainfluenza virus (multiple types), avian influenza (various types), shipping fever virus, Western and Eastern equine encephalomyelitis, Japanese encephalomyelitis, fowl pox, rabies virus, slow brain viruses, rous sarcoma virus, Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), Togaviridae (e.g., Rubivirus), Newcastle disease virus, West Nile fever virus, Tick borne encephalitis, yellow fever, chikungunya virus, and dengue virus (all serotypes).

In another embodiment, the specific proteins from viruses may comprise: HA and/or NA from influenza virus (including avian), S protein from coronavirus, gp160, gp140 and/or gp41 from HIV, gp I to IV and Vp from varicella zoster, E and preM/M from yellow fever virus, Dengue (all serotypes) or any flavivirus. Also included are any protein from a virus that can induce an immune response (cellular and/or humoral) in a vertebrate that can prevent, treat, manage and/or ameliorate an infectious disease in said vertebrate.

Non-limiting examples of bacteria from which said infectious agent proteins can be derived from are the following: B. pertussis, Leptospira pomona, S. paratyphi A and B, C. diphtheriae, C. tetani, C. botulinum, C. perfringens, C. feseri and other gas gangrene bacteria, B. anthracis, P. pestis, P. multocida, Neisseria meningitidis, N. gonorrheae, Hemophilus influenzae, Actinomyces (e.g., Norcardia), Acinetobacter, Bacillaceae (e.g., Bacillus anthrasis), Bacteroides (e.g., Bacteroides fragilis), Blastomycosis, Bordetella, Borrelia (e.g., Borrelia burgdorferi), Brucella, Campylobacter, Chlamydia, Coccidioides, Corynebacterium (e.g., Corynebacterium diptheriae), E. coli (e.g., Enterotoxigenic E. coli and Enterohemorrhagic E. coli), Enterobacter (e.g. Enterobacter aerogenes), Enterobacteriaceae (Klebsiella, Salmonella (e.g., Salmonella typhi, Salmonella enteritidis, Serratia, Yersinia, Shigella), Erysipelothrix, Haemophilus (e.g., Haemophilus influenza type B), Helicobacter, Legionella (e.g., Legionella pneumophila), Leptospira, Listeria (e.g., Listeria monocytogenes), Mycoplasma, Mycobacterium (e.g., Mycobacterium leprae and Mycobacterium tuberculosis), Vibrio (e.g., Vibrio cholerae), Pasteurellacea, Proteus, Pseudomonas (e.g., Pseudomonas aeruginosa), Rickettsiaceae, Spirochetes (e.g., Treponema spp., Leptospira spp., Borrelia spp.), Shigella spp., Meningiococcus, Pneumococcus and Streptococcus (e.g., Streptococcus pneumoniae and Groups A, B, and C Streptococci), Ureaplasmas. Treponema pollidum, Staphylococcus aureus, Pasteurella haemolytica, Corynebacterium diptheriae toxoid, Meningococcal polysaccharide, Bordetella pertusis, Streptococcus pneumoniae, Clostridium tetani toxoid, and Mycobacterium bovis.

Non-limiting examples of parasites from which said infectious agent proteins can be derived from are the following: leishmaniasis (Leishmania tropica mexicana, Leishmania tropica, Leishmania major, Leishmania aethiopica, Leishmania braziliensis, Leishmania donovani, Leishmania infantum, Leishmania chagasi), trypanosomiasis (Trypanosoma brucei gambiense, Trypanosoma brucei rhodesiense), toxoplasmosis (Toxoplasma gondii), schistosomiasis (Schistosoma haematobium, Schistosoma japonicum, Schistosoma mansoni, Schistosoma mekongi, Schistosoma intercalatum), malaria (Plasmodium virax, Plasmodium falciparium, Plasmodium malariae and Plasmodium ovale) Amebiasis (Entamoeba histolytica), Babesiosis (Babesiosis microti), Cryptosporidiosis (Cryptosporidium parvum), Dientamoebiasis (Dientamoeba fragilis), Giardiasis (Giardia lamblia), Helminthiasis and Trichomonas (Trichomonas vaginalis). The above lists are meant to be illustrative and by no means are meant to limit the invention to those particular bacterial, viral or parasitic organisms.

The invention also encompasses variants of the said proteins expressed on or in the VLPs of the invention. The variants may contain alterations in the amino acid sequences of the constituent proteins. The term “variant” with respect to a protein refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

Natural variants can occur due to mutations in the proteins. These mutations may lead to antigenic variability within individual groups of infectious agents, for example influenza. Thus, a person infected with an influenza strain develops antibody against that virus, as newer virus strains appear, the antibodies against the older strains no longer recognize the newer virus and reinfection can occur. The invention encompasses all antigenic and genetic variability of proteins from infectious agents for making VLPs.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating F and/or G molecules of RSV, etc. Thus, the invention also encompasses using known methods of protein engineering and recombinant DNA technology to improve or alter the characteristics of the proteins expressed on or in the VLPs of the invention. Various types of mutagenesis can be used to produce and/or isolate variant nucleic acids that encode for protein molecules and/or to further modify/mutate the proteins in or on the VLPs of the invention. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, is also included in the present invention. In one embodiment, mutagenesis can be guided by known information of the naturally occurring molecule or altered or mutated naturally occurring molecule, e.g., sequence, sequence comparisons, physical properties, crystal structure or the like.

The invention further comprises protein variants which show substantial biological activity, e.g., able to elicit an effective antibody response when expressed on or in VLPs of the invention. Such variants include deletions, insertions, inversions, repeats, and substitutions selected according to general rules known in the art so as have little effect on activity. An example of a mutation is to remove the cleavage site in the RSV F protein and/or remove or add a glycosylation site.

Methods of cloning said proteins are known in the art. For example, the gene encoding a specific RSV protein can be isolated by RT-PCR from polyadenylated mRNA extracted from cells which had been infected with a RSV virus. The resulting product gene can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids or bacmids.

Thus, the invention comprises nucleotides that encode proteins, including chimeric molecules, cloned into an expression vector that can be expressed in a cell that induces the formation of VLPs of the invention. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, said nucleotides encode for a chimeric RSV F protein (as discussed above). In another embodiment, said vector comprises nucleotides that encode the F and/or M RSV proteins. In another embodiment, said vector comprises nucleotides that encode the F, G, M and/or N RSV proteins. In another embodiment, said vector comprises nucleotides that encode the chimeric RSV F, M protein or optionally influenza M1 protein. In another embodiment, the expression vector is a baculovirus vector.

The invention also utilizes nucleic acid and polypeptides which encode RSV F, G and M. In one embodiment, a RSV F nucleic acid or protein is at least 85%, 90%, 95%, 96%, 97%, 98% or 99% identical to SEQ ID NOs 3 or 4, respectively. In another embodiment, RSV G nucleic acid or protein is at least 85%, 90%, 95%, 96%, 97%, 98% or 99% identical to SEQ ID NOs 2 or 5, respectively. In another embodiment, RSV M nucleic acid or protein is at least 85%, 90%, 95%, 96%, 97%, 98% or 99% identical to SEQ ID NOs 1 or 6, respectively.

The invention also includes all the chimeric molecules made from F proteins. In one embodiment, a chimeric F protein nucleic acid or protein is at least 85%, 90%, 95%, 96%, 97%, 98% or 99% identical to SEQ ID NOs 9, 10, 11 and/or 12 respectively.

In some embodiments of the invention proteins may comprise, mutations containing alterations which produce silent substitutions, additions, or deletions, but do not alter the properties or activities of the encoded protein or how the proteins are made. Nucleotide variants can be produced for a variety of reasons, e.g., to optimize codon expression for a particular host (change codons in the human mRNA to those preferred by insect cells such as Sf9 cells, see SEQ ID Nos. 4, 5 and 6). See U.S. patent publication 2005/0118191, herein incorporated by reference in its entirety for all purposes.

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the RSV viral proteins can be cloned. A person with skill in the art understands that additional methods are available and are possible.

The invention also provides for constructs and/or vectors that comprise RSV nucleotides that encode for RSV structural genes, including F, G, M, N or portions thereof, and/or any chimeric molecule described above. The vector may be, for example, a phage, plasmid, viral, or retroviral vector. The constructs and/or vectors that comprise RSV structural genes, including F, G, M, N or portions thereof, and/or any chimeric molecule described above, should be operatively linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan. In one embodiment, said vector that comprise nucleotides encoding for RSV genes, including F, G, M, N or portions thereof, and/or any chimeric molecule described above, is pFastBac. In another embodiment, said vector that comprises an insert that consists of nucleotides encoding for RSV genes, comprising F and M, is pFastBac. In another embodiment, said vector that comprises an insert that consists of nucleotides encoding for RSV genes, comprising RSV F, G and M, is pFastBac. In another embodiment, said vector that comprises an insert that consists of nucleotides encoding for RSV genes, comprising RSV F, G, M, and/or N is pFastBac. In another embodiment, said vector that comprises an insert that consists of nucleotides encoding for RSV genes, comprising RSV F, M, and N is pFastBac. In another embodiment, said vector that comprises an insert that consists of nucleotides encoding for RSV genes, comprising chimeric RSV F and optionally influenza M1, is pFastBac. In another embodiment, said vector that comprises an insert that comprises nucleotides encoding for RSV M protein and at least one protein from another infectious agent, is pFastBac. In another embodiment, said vector that comprises an insert that consists of nucleotides encoding for RSV M protein and at least one protein from another infectious agent, is pFastBac. In another embodiment, said vector that comprises an insert that comprise SEQ ID NOs 5, 6, 7, 8, 10, and/or 12 is pFastBac. In another embodiment, said vector that comprises an insert that consists of SEQ ID NOs 5, 6, 7, 8, 10, and/or 12 is pFastBac.

Next, the recombinant constructs mentioned above could be used to transfect, infect, or transform and can express RSV proteins, including F, G, M, N, or portions thereof, and/or any chimeric molecule described above, into eukaryotic cells and/or prokaryotic cells. Thus, the invention provides for host cells which comprise a vector (or vectors) that contain nucleic acids which code for RSV structural genes, including F, G, M, N, or portions thereof, and/or any chimeric molecule described above, and permit the expression of RSV structural genes, including F, G, M, N, or portions thereof, and/or any chimeric molecule described above in said host cell under conditions which allow the formation of VLPs.

Among eukaryotic host cells are yeast, insect, avian, plant, C. elegans (or nematode) and mammalian host cells. Non limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria.

Vectors, e.g., vectors comprising polynucleotides of RSV F, G, M, N, or portions thereof, and/or any chimeric molecule described above, can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, said vector is a recombinant baculovirus. In another embodiment, said recombinant baculovirus is transfected into a eukaryotic cell. In a preferred embodiment, said cell is an insect cell. In another embodiment, said insect cell is a Sf9 cell.

In another embodiment, said vector and/or host cell comprise nucleotides that encode RSV genes, including F, G, M, or portions thereof, and/or any chimeric molecule described above. In another embodiment, said vector and/or host cell consists essentially of RSV F, G, M, N, or portions thereof, and/or any chimeric molecule described above. In a further embodiment, said vector and/or host cell consists of RSV protein comprising F, G, M, N, or portions thereof, and/or any chimeric molecule described above. These vector and/or host cell contain RSV F, G, M, N, or portions thereof, and/or any chimeric molecule described above, and may contain additional cellular constituents such as cellular proteins, baculovirus proteins, lipids, carbohydrates etc., but do not contain additional RSV proteins (other than fragments of RSV F, G, M, N, or portions thereof, and/or any chimeric molecule described above).

This invention also provides for constructs and methods that will increase the efficiency of VLPs production. For example, the addition of leader sequences to the RSV F, G, M, N, or portions thereof, and/or any chimeric molecule described above, can improve the efficiency of protein transporting within the cell. For example, a heterologous signal sequence can be fused to the RSV F, G, M, N, or portions thereof, and/or any chimeric molecule described above. In one embodiment, the signal sequence can be derived from the gene of an insect cell and fused to RSV F, G, M, N, or portions thereof, and/or any chimeric molecule described above. In another embodiment, the signal peptide is the chitinase signal sequence, which works efficiently in baculovirus expression systems.

Another method to increase efficiency of VLP production is to codon optimize the nucleotides that encode RSV including F, G, M, N or portions thereof, and/or any chimeric molecule described above for a specific cell type. For examples of codon optimizing nucleic acids for expression in Sf9 cell see SEQ ID Nos. 4, 5 and 6 and U.S. patent publication 2005/0118191, herein incorporated by reference in its entirety for all purposes.

The invention also provides for methods of producing VLPs, said methods comprising expressing RSV genes including F, G, M, N, or portions thereof, and/or any chimeric molecule described above under conditions that allow VLP formation. Depending on the expression system and host cell selected, the VLPs are produced by growing host cells transformed by an expression vector under conditions whereby the recombinant proteins are expressed and VLPs are formed. In one embodiment, the invention comprises a method of producing a VLP, comprising transfecting vectors encoding at least one RSV M protein into a suitable host cell and expressing said RSV virus protein under conditions that allow VLP formation. In another embodiment, said eukaryotic cell is selected from the group consisting of, yeast, insect, amphibian, avian or mammalian cells. The selection of the appropriate growth conditions is within the skill or a person with skill of one of ordinary skill in the art.

Methods to grow cells engineered to produce VLPs of the invention include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored. In one embodiment, said bioreactor is a stainless steel chamber. In another embodiment, said bioreactor is a pre-sterilized plastic bag (e.g. Cellbag®, Wave Biotech, Bridgewater, N.J.). In other embodiment, said pre-sterilized plastic bags are about 50 L to 1000 L bags.

The VLPs are then isolated using methods that preserve the integrity thereof, such as by gradient centrifugation, e.g., cesium chloride, sucrose and iodixanol, as well as standard purification techniques including, e.g., ion exchange and gel filtration chromatography.

The following is an example of how VLPs of the invention can be made, isolated and purified. Usually VLPs are produced from recombinant cell lines engineered to create VLPs when said cells are grown in cell culture (see above). A person of skill in the art would understand that there are additional methods that can be utilized to make and purify VLPs of the invention, thus the invention is not limited to the method described.

Production of VLPs of the invention can start by seeding Sf9 cells (non-infected) into shaker flasks, allowing the cells to expand and scaling up as the cells grow and multiply (for example from a 125-ml flask to a 50 L Wave bag). The medium used to grow the cell is formulated for the appropriate cell line (preferably serum free media, e.g. insect medium ExCell-420, JRH). Next, said cells are infected with recombinant baculovirus at the most efficient multiplicity of infection (e.g. from about 1 to about 3 plaque forming units per cell). Once infection has occurred, the RSV F, G, M, N, or portions thereof, and/or any chimeric molecule described above, are expressed from the virus genome, self assemble into VLPs and are secreted from the cells approximately 24 to 72 hours post infection. Usually, infection is most efficient when the cells are in mid-log phase of growth (4-8×10⁶ cells/ml) and are at least about 90% viable.

VLPs of the invention can be harvested approximately 48 to 96 hours post infection, when the levels of VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the invention can be accomplished by tangential flow filtration (TFF) with a single use, pre-sterilized hollow fiber 0.5 or 1.00 μm filter cartridge or a similar device.

Next, VLPs in the clarified culture medium can be concentrated by ultrafiltration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components.

The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually VLPs will form a distinctive visible band between about 30% to about 40% sucrose or at the interface (in a 20% and 60% step gradient) that can be collected from the gradient and stored. This product can be diluted to comprise 200 mM of NaCl in preparation for the next step in the purification process. This product contains VLPs and may contain intact baculovirus particles.

Further purification of VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluded via a salt gradient (from about 0.2 M to about 1.0 M of NaCl) that can separate the VLP from other contaminates (e.g. baculovirus and DNA/RNA). In the sucrose cushion method, the sample comprising the VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The VLP peak or band is collected.

The intact baculovirus can be inactivated, if desired. Inactivation can be accomplished by chemical methods, for example, formalin or β-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour.

After the inactivation/removal step, the product comprising VLPs can be run through another diafiltration step to remove any reagent from the inactivation step and/or any residual sucrose, and to place the VLPs into the desired buffer (e.g. PBS). The solution comprising VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

Expansion and production of baculovirus expression vectors and infection of cells with recombinant baculovirus to produce recombinant RSV VLPs can be accomplished in insect cells, for example Sf9 insect cells as previously described. In one embodiment, the cells are SF9 infected with recombinant baculovirus engineered to produce RSV VLPs.

### Pharmaceutical or Vaccine Formulations and Administration

The pharmaceutical compositions useful herein contain a pharmaceutically acceptable carrier, including any suitable diluent or excipient, which includes any pharmaceutical agent that does not itself induce the production of an immune response harmful to the vertebrate receiving the composition, and which may be administered without undue toxicity and a VLP of the invention. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic compositions for inducing a protective immune response in a vertebrate.

The invention encompasses an antigenic formulation comprising VLPs which comprises at least one RSV protein. In one embodiment, said antigenic formulation comprises VLPs comprising RSV F protein. In another embodiment, said antigenic formulation comprises VLPs comprising RSV M protein. In another embodiment, said antigenic formulation comprises VLPs further comprising RSV G protein. In another embodiment, said antigenic formulation comprises VLPs further comprising RSV N protein. In another embodiment, said antigenic formulation comprises VLPs comprising the G protein from RSV group A. In another embodiment, said antigenic formulation comprises VLPs comprising the G protein from RSV group B. In another embodiment, the invention encompasses an antigenic formulation comprising chimeric VLPs such as VLPs comprising chimeric F protein from a RSV and optionally M protein derived from an influenza virus, wherein said chimeric F protein is a fused to the transmembrane domain and cytoplasmic tail of influenza HA protein.

The invention also encompasses an antigenic formulation comprising a chimeric VLP that comprises at least one RSV protein. In one embodiment, the antigenic formulation comprises VLPs comprising a RSV M protein and at least one protein from another infectious agent. In another embodiment, said protein from another infectious agent is a viral protein. In another embodiment, said protein from an infectious agent is an envelope associated protein. In another embodiment, said protein from another infectious agent is expressed on the surface of VLPs. In another embodiment, said protein from an infectious agent comprises an epitope that will generate a protective immune response in a vertebrate. In another embodiment, said protein from another infectious agent can associated with RSV M protein. In another embodiment, said protein from another infectious agent is fused to a RSV protein. In another embodiment, only a portion of a protein from another infectious agent is fused to a RSV protein. In another embodiment, only a portion of a protein from another infectious agent is fused to a portion of a RSV protein. In another embodiment, said portion of the protein from another infectious agent fused to said RSV protein is expressed on the surface of VLPs. In other embodiment, said RSV protein, or portion thereof, fused to the protein from another infectious agent associates with the RSV M protein. In other embodiment, said RSV protein, or portion thereof, is derived from RSV F, G, N and/or P. In another embodiment, said chimeric VLPs further comprise N and/or P protein from RSV. In another embodiment, said chimeric VLPs comprise more than one protein from an infectious agent. In another embodiment, said chimeric VLPs comprise more one infectious agent proteins, thus creating a multivalent VLP.

The invention also encompasses a vaccine formulation comprising VLPs that comprise at least one RSV protein. In one embodiment, said vaccine formulation comprises VLPs comprising a RSV F protein. In one embodiment, said vaccine formulation comprises VLPs comprising a RSV M protein. In another embodiment, said vaccine formulation comprises VLPs further comprising a RSV G protein. In another embodiment, said vaccine formulation comprises VLPs comprising the G protein from RSV group A. In another embodiment, said vaccine formulation comprises VLPs comprising the G protein from RSV group B. In another embodiment, the invention encompasses a vaccine formulation comprising VLPs which comprises a chimeric F protein from a RSV and optionally M1 protein derived from an influenza virus, wherein said chimeric F protein is a fused to the transmembrane domain and cytoplasmic tail of influenza HA protein.

The invention also encompasses a vaccine formulation comprising chimeric VLPs that comprise at least one RSV protein. In one embodiment, the vaccine formulation comprises VLPs comprising a RSV M protein and at least one protein from another infectious agent. In another embodiment, said protein from another infectious agent is a viral protein. In another embodiment, said protein from an infectious agent is an envelope associated protein. In another embodiment, said protein from another infectious agent is expressed on the surface of VLPs. In another embodiment, said protein from an infectious agent comprises an epitope that will generate a protective immune response in a vertebrate. In another embodiment, said protein from another infectious agent can associated with RSV M protein. In another embodiment, said protein from another infectious agent is fused to a RSV protein. In another embodiment, only a portion of a protein from another infectious agent is fused to a RSV protein. In another embodiment, only a portion of a protein from another infectious agent is fused to a portion of a RSV protein. In another embodiment, said portion of the protein from another infectious agent fused to said RSV protein is expressed on the surface of VLPs. In other embodiment, said RSV protein, or portion thereof, fused to the protein from another infectious agent associates with the RSV M protein. In other embodiment, said RSV protein, or portion thereof, is derived from RSV F, G, N and/or P. In another embodiment, said chimeric VLPs further comprise N and/or P protein from RSV. In another embodiment, said chimeric VLPs comprise more than one protein from an infectious agent. In another embodiment, said chimeric VLPs comprise more one infectious agent proteins, thus creating a multivalent VLP.

Said formulations of the invention comprise VLPs comprising RSV F, G, M, N, or portions thereof, and/or any chimeric molecule described above and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention also provides for a pharmaceutical pack or kit comprising one or more containers filled with one or more of the ingredients of the vaccine formulations of the invention. In a preferred embodiment, the kit comprises two containers, one containing VLPs and the other containing an adjuvant. Associated with such container(s) can be a notice in the form prescribed by a governmental agency regulating the manufacture, use or sale of pharmaceuticals or biological products, which notice reflects approval by the agency of manufacture, use or sale for human administration.

The invention also provides that the VLP formulation be packaged in a hermetically sealed container such as an ampoule or sachette indicating the quantity of composition. In one embodiment, the VLP composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the VLP composition is supplied in liquid form in a hermetically sealed container indicating the quantity and concentration of the VLP composition. Preferably, the liquid form of the VLP composition is supplied in a hermetically sealed container at least about 50 μg/ml, more preferably at least about 100 μg/ml, at least about 200 μg/ml, at least 500 μg/ml, or at least 1 mg/ml.

Generally, RSV VLPs of the invention are administered in an effective amount or quantity (as defined above) sufficient to stimulate an immune response against one or more strains of RSV. Preferably, administration of the VLP of the invention elicits immunity against RSV. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in an immune response, intranasal administration confers the added benefit of eliciting mucosal immunity at the site of entry of many viruses, including RSV and influenza.

Thus, the invention also comprises a method of formulating a vaccine or antigenic composition that induces immunity to an infection or at least one symptom thereof to a mammal, comprising adding to said formulation an effective dose of RSV VLPs. In one embodiment, said infection is an RSV infection.

While stimulation of immunity with a single dose is preferred, additional dosages can be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections, e.g. RSV infection. Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Methods of administering a composition comprising VLPs (vaccine and/or antigenic formulations) include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered intramuscularly, intravenously, subcutaneously, transdermally or intradermally. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition comprising VLPs of the invention may induce an antibody or other immune response that is substantially higher than other routes of administration. In another embodiment, intranasal or other mucosal routes of administration of a composition comprising VLPs of the invention may induce an antibody or other immune response that will induce cross protection against other strains of RSV. Administration can be systemic or local.

In yet another embodiment, the vaccine and/or antigenic formulation is administered in such a manner as to target mucosal tissues in order to elicit an immune response at the site of immunization. For example, mucosal tissues such as gut associated lymphoid tissue (GALT) can be targeted for immunization by using oral administration of compositions which contain adjuvants with particular mucosal targeting properties. Additional mucosal tissues can also be targeted, such as nasopharyngeal lymphoid tissue (NALT) and bronchial-associated lymphoid tissue (BALT).

Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, said VLPs of the invention can be administered as part of a combination therapy. For example, VLPs of the invention can be formulated with other immunogenic compositions, antivirals and/or antibiotics.

The dosage of the pharmaceutical formulation can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. Said dosages can be determined from animal studies. A non-limiting list of animals used to study the efficacy of vaccines include the guinea pig, hamster, ferrets, chinchilla, mouse and cotton rat. Most animals are not natural hosts to infectious agents but can still serve in studies of various aspects of the disease. For example, any of the above animals can be dosed with a vaccine candidate, e.g. VLPs of the invention, to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants. Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention.

Exemplary, adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MF-59, Novasomes®, MHC antigens may also be used.

In one embodiment of the invention the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes).

The VLPs of the invention can also be formulated with “immune stimulators.” These are the body's own chemical messengers (cytokines) to increase the immune system's response. Immune stimulators include, but not limited to, various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immunostimulatory molecules can be administered in the same formulation as the RSV VLPs, or can be administered separately. Either the protein or an expression vector encoding the protein can be administered to produce an immunostimulatory effect. Thus in one embodiment, the invention comprises antigentic and vaccine formulations comprising an adjuvant and/or an immune stimulator.

### Methods of Stimulating an Immune Response

The VLPs of the invention are useful for preparing compositions that stimulate an immune response that confers immunity or substantial immunity to infectious agents. Both mucosal and cellular immunity may contribute to immunity to infectious agents and disease. Antibodies secreted locally in the upper respiratory tract are a major factor in resistance to natural infection. Secretory immunoglobulin A (sIgA) is involved in protection of the upper respiratory tract and serum IgG in protection of the lower respiratory tract. The immune response induced by an infection protects against reinfection with the same virus or an antigenically similar viral strain. For example, RSV undergoes frequent and unpredictable changes; therefore, after natural infection, the effective period of protection provided by the host's immunity may only be a few years against the new strains of virus circulating in the community.

Thus, the invention encompasses a method of inducing immunity to infections or at least one symptom thereof in a subject, comprising administering at least one effective dose of RSV VLPs. In another embodiment, said method comprises administering VLPs comprising RSV F protein. In another embodiment, said method comprises administering VLPs comprising RSV M protein. In another embodiment, said method comprises administering VLPs further comprising RSV G protein. In another embodiment, said method comprises administering VLPs comprising the G protein from RSV group A or group B. In another embodiment, said method comprises administering VLPs comprising chimeric F protein from RSV and optionally M protein derived from an influenza virus, wherein said chimeric F protein is fused to the transmembrane domain and cytoplasmic tail of influenza HA protein. In another embodiment, said subject is a mammal. In another embodiment, said mammal is a human. In another embodiment, RSV VLPs are formulated with an adjuvant or immune stimulator.

Another embodiment of the invention comprises a method to induce immunity to RSV infection or at least one symptom thereof in a subject, comprises administering at least one effective dose of a RSV VLPs, wherein said VLPs comprise RSV F (including chimeric F), G, N and/or M (or optionally M1 from influenza) proteins. In another embodiment, a method of inducing immunity to RSV infection or at least one symptom thereof in a subject, comprises administering at least one effective dose of a RSV VLPs, wherein said VLPs consists essentially of RSV F (including chimeric F), G, N and/or M (or optionally M1 from influenza) proteins. Said VLPs may comprise additional RSV proteins and/or protein contaminates in negligible concentrations. In another embodiment, a method of inducing immunity to RSV infection or at least one symptom thereof in a subject, comprises administering at least one effective dose of a RSV VLPs, wherein said VLPs consists of RSV F (including chimeric F), G and/or M (or optionally M1 from influenza). In another embodiment, a method of inducing immunity to RSV infection or at least one symptom thereof in a subject, comprises administering at least one effective dose of a RSV VLPs comprising RSV proteins, wherein said RSV proteins consist of RSV F (including chimeric F), G, N and/or M (or optionally M1 from influenza) proteins. These VLPs contain RSV F (including chimeric F), G, N and/or M (or optionally M1 from influenza) proteins and may contain additional cellular constituents such as cellular proteins, baculovirus proteins, lipids, carbohydrates etc., but do not contain additional RSV proteins (other than fragments of RSV F (including chimeric F), G, N and/or M (or optionally M1 from influenza) proteins). In another embodiment, said subject is a mammal. In another embodiment, said mammal is a human. In another embodiment, the method comprises inducing immunity to RSV infection or at least one symptom thereof by administering said formulation in one dose. In another embodiment, the method comprises inducing immunity to RSV infection or at least one symptom thereof by administering said formulation in multiple doses.

The invention also encompasses inducing immunity to an infection, or at least one symptom thereof, in a subject caused by an infectious agent, comprising administering at least one effective dose of chimeric VLPs of the invention. In one embodiment, said method comprises administering VLPs comprising a RSV M protein and at least one protein from another infectious agent. In another embodiment, said protein from another infectious agent is a viral protein. In another embodiment, said protein from an infectious agent is an envelope associated protein. In another embodiment, said protein from another infectious agent is expressed on the surface of VLPs. In another embodiment, said protein from an infectious agent comprises an epitope that will generate a protective immune response in a vertebrate. In another embodiment, said protein from another infectious agent can associated with RSV M protein. In another embodiment, said protein from another infectious agent is fused to a RSV protein. In another embodiment, only a portion of a protein from another infectious agent is fused to a RSV protein. In another embodiment, only a portion of a protein from another infectious agent is fused to a portion of a RSV protein. In another embodiment, said portion of the protein from another infectious agent fused to said RSV protein is expressed on the surface of VLPs. In other embodiment, said RSV protein, or portion thereof, fused to the protein from another infectious agent associates with the RSV M protein. In other embodiment, said RSV protein, or portion thereof, is derived from RSV F, G, N and/or P. In another embodiment, said chimeric VLPs further comprise N and/or P protein from RSV. In another embodiment, said chimeric VLPs comprise more than one protein from an infectious agent. In another embodiment, said chimeric VLPs comprise more one infectious agent protein, thus creating a multivalent VLP.

VLPs of the invention can induce substantial immunity in a vertebrate (e.g. a human) when administered to said vertebrate. The substantial immunity results from an immune response against VLPs of the invention that protects or ameliorates infection or at least reduces a symptom of infection in said vertebrate. In some instances, if the said vertebrate is infected, said infection will be asymptomatic. The response may be not a fully protective response. In this case, if said vertebrate is infected with an infectious agent, the vertebrate will experience reduced symptoms or a shorter duration of symptoms compared to a non-immunized vertebrate.

In one embodiment, the invention comprises a method of inducing substantial immunity to RSV virus infection or at least one symptom thereof in a subject, comprising administering at least one effective dose of RSV VLPs. In another embodiment, the invention comprises a method of vaccinating a mammal against RSV comprising administering to said mammal a protection-inducing amount of VLPs comprising at least one RSV protein. In one embodiment, said method comprises administering VLPs comprising RSV F protein. In another embodiment, said method comprises administering VLPs comprising RSV M protein. In another embodiment, said method comprises administering VLPs comprising the G protein from RSV group A. In another embodiment, said method comprises administering VLPs comprising the G protein from RSV group B. In another embodiment, said method comprises administering VLPs comprising chimeric F protein from RSV and M protein derived from an influenza virus, wherein said chimeric F protein is a fused to the transmembrane domain and cytoplasmic tail of influenza HA protein.

The invention also encompasses a method of inducing substantial immunity to an infection, or at least one symptom thereof, in a subject caused by an infectious agent, comprising administering at least one effective dose of chimeric VLPs of the invention. In one embodiment, said method comprises administering VLPs comprising a RSV M protein and at least one protein from another infectious agent. In another embodiment, said protein from another infectious agent is a viral protein. In another embodiment, said protein from an infectious agent is an envelope associated protein. In another embodiment, said protein from another infectious agent is expressed on the surface of VLPs. In another embodiment, said protein from an infectious agent comprises an epitope that will generate a protective immune response in a vertebrate. In another embodiment, said protein from another infectious agent can associated with RSV M protein. In another embodiment, said protein from another infectious agent is fused to a RSV protein. In another embodiment, only a portion of a protein from another infectious agent is fused to a RSV protein. In another embodiment, only a portion of a protein from another infectious agent is fused to a portion of a RSV protein. In another embodiment, said portion of the protein from another infectious agent fused to said RSV protein is expressed on the surface of VLPs. In other embodiment, said RSV protein, or portion thereof, fused to the protein from another infectious agent associates with the RSV M protein. In other embodiment, said RSV protein, or portion thereof, is derived from RSV F, G, N and/or P. In another embodiment, said chimeric VLPs further comprise N and/or P protein from RSV. In another embodiment, said chimeric VLPs comprise more than one protein from an infectious agent. In another embodiment, said chimeric VLPs comprise more one infectious agent protein, thus creating a multivalent VLP.

In another embodiment, the invention comprises a method of inducing a protective antibody response to an infection or at least one symptom thereof in a subject, comprising administering at least one effective dose of RSV VLPs, wherein said VLPs comprises RSV including F, G, M, N, or portions thereof, and/or any chimeric molecule described above.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. Antibodies exist as intact immunoglobulins or as a number of well-characterized fragments produced by digestion with various peptidases.

In another embodiment, the invention comprises a method of inducing a protective cellular response to RSV infection or at least one symptom thereof in a subject, comprising administering at least one effective dose of a RSV VLP, wherein said VLP comprises including F, G, M, N or portions thereof, and/or any chimeric molecule described above. Cell-mediated immunity also plays a role in recovery from RSV infection and may prevent RSV-associated complications. RSV-specific cellular lymphocytes have been detected in the blood and the lower respiratory tract secretions of infected subjects. Cytolysis of RSV-infected cells is mediated by CTLs in concert with RSV-specific antibodies and complement. The primary cytotoxic response is detectable in blood after 6-14 days and disappears by day 21 in infected or vaccinated individuals (Ennis et al., 1981). Cell-mediated immunity also plays a role in recovery from RSV infection and may prevent RSV-associated complications. RSV-specific cellular lymphocytes have been detected in the blood and the lower respiratory tract secretions of infected subjects.

As mentioned above, the VLPs of the invention prevent or reduce at least one symptom of RSV infection in a subject. Symptoms of RSV are well known in the art. They include rhinorrhea, sore throat, headache, hoarseness, cough, sputum, fever, rales, wheezing, and dyspnea. Thus, the method of the invention comprises the prevention or reduction of at least one symptom associated with RSV infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a RSV infection or additional symptoms, a reduced severity of a RSV symptoms or a suitable assays (e.g. antibody titer and/or T-cell activation assay). The objective assessment comprises both animal and human assessments.

This invention is further illustrated by the following examples that should not be construed as limiting. The contents of all references, patents and published patent applications cited throughout this application, as well as the Figures and the Sequence Listing, are incorporated herein by reference for all purposes.

### EXAMPLES

### Example 1

### Generating the Recombinant Bacmids

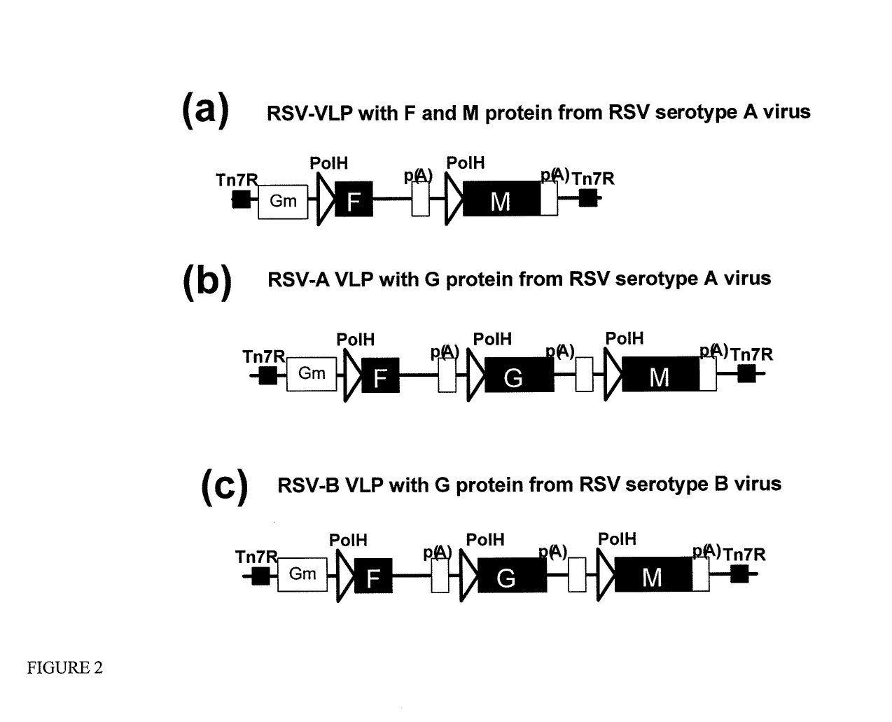

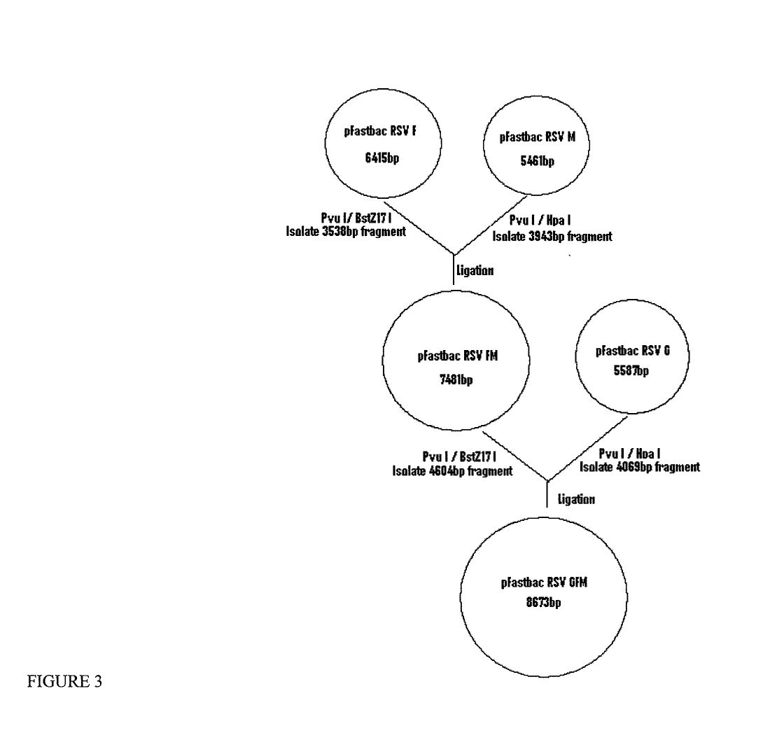

RSV-VLPs were generated with the M protein of RSV alone and in combination with RSV G protein. Additional constructs comprise RSV fusion (F) alone and in combination with RSV G and M. The protein sequences below were used to synthesize genes in which the nucleotides were codon optimized for insect cells and the genes were cloned into bacmids. A general representation of the constructs is illustrated on FIG. 2 and the cloning strategy for making the constructs is illustrated in FIG. 3.

Once the desired constructs were confirmed and purified, one vial of MAX Efficiency® DH10Bac™ competent cells for each construct was thawed on ice. Approximately 1 ng (5 μl) of the desired pFastBac™ construct plasmid DNA was added to the cells and mixed gently. The cells were incubated on ice for 30 minutes. This was followed by heat-shock of the cells for 45 seconds at 42° C. without shaking. Next, the tubes where immediately transferred to ice and chilled for 2 minutes. Subsequently 900 μl of room temperature S.O.C. Medium was added to each tube. The tubes where put on a shaker at 37° C. at 225 rpm for 4 hours. For each pFastBac™ transformation, 10-fold serial dilutions of the cells (10⁻¹, 10⁻² and 10⁻³) was prepared using S.O.C. medium. Next, 100 μl of each dilution was plated on an LB agar plate containing 50 μg/ml kanamycin, 7 μg/ml gentamicin, μg/ml tetracycline, 100 μg/ml Bluo-gal, and 40 μg/ml IPTG. The plates were incubated for 48 hours at 37° C. White colonies where picked for analysis.

RSV (applicable for Type A & B) Protein Sequence:
RSV M (SEQ ID NO. 1)
METYVNKLHEGSTYTAAVQYNVLEKDDDPASLTIWVPMFQSSMPADLLIK
ELANVNILVKQISTPKGPSLRVMINSRSAVLAQMPSKFTICANVSLDERS
KLAYDVTTPCEIKACSLTCLKSKNMLTTVKDLTMKTLNPTHDIIALCEFE
NIVTSKKVIIPTYLRSISVRNKDLNTLENITTTEFKNAITNAKIIPYSGL
LLVITVTDNKGAFKYIKPQSQFIVDLGAYLEKESIYYVTTNWKHTATRFA
IKPMED*
RSV G (SEQ ID NO. 2)
MSKNKDQRTAKTLERTWDTLNHLLFISSCLYKLNLKSVAQITLSILAMII
STSLIIAAIIFIASANHKVTPTTAIIQDATSQIKNTTPTYLTQNPQLGIS
PSNPSEITSQITTILASTTPGVKSTLQSTTVKTKNTTTTQTQPSKPTTKQ
RQNKPPSKPNNDFHFEVFNFVPCSICSNNPTCWAICKRIPNKKPGKKTTT
KPTKKPTLKTTKKDPKPQTTKSKEVPTTKPTEEPTINTTKTNIITTLLTS
NTTGNPELTSQMETFHSTSSEGNPSPSQVSTTSEYPSQPSSPPNTPRQ*
RSV F (SEQ ID NO. 3)
MELLILKANAITTILTAVTFCFASGQNITEEFYQSTCSAVSKGYLSALRT
GWYTSVITIELSNIKENKCNGTDAKVKLIKQELDKYKNAVTELQLLMQST
PPTNNRARRELPRFMNYTLNNAKKTNVTLSKKRKRRFLGFLLGVGSAIAS
GVAVSKVLHLEGEVNKIKSALLSTNKAVVSLSNGVSVLTSKVLDLKNYID
KQLLPIVNKQSCSISNIETVIEFQQKNNRLLEITREFSVNAGVTTPVSTY
MLTNSELLSLINDMPITNDQKKLMSNNVQIVRQQSYSIMSIIKEEVLAYV
VQLPLYGVIDTPCWKLHTSPLCTTNTKEGSNICLTRTDRGWYCDNAGSVS
FFPQAETCKVQSNRVFCDTMNSLTLPSEINLCNVDIFNPKYDCKIMTSKT
DVSSSVITSLGAIVSCYGKTKCTASNKNRGIIKTFSNGCDYVSNKGMDTV
SVGNTLYYVNKQEGKSLYVKGEPIINFYDPLVFPSDEFDASISQVNEKIN
QSLAFIRKSDELLHNVNAGKSTTNIMITTIIIVIIVILLSLIAVGLLLYC
KARSTPVTLSKDQLSGINNIAFSN*
RSV N (SEQ ID NO. 4)
MALSKVKLNDTLNKDQLLSSSKYTIQRSTGDSIDTPNYDVQKHINKLCGM
LLITEDANHKFTGLIGMLYAMSRLGREDTIKILRDAGYHVKANGVDVTTH
RQDINGKEMKFEVLTLASLTTEIQINIEIESRKSYKKMLKEMGEVAPEYR
HDSPDCGMIILCIAALVITKLAAGDRSGLTAVIRRANNVLKNEMKRYKGL
LPKDIANSFYEVFEKHPHFIDVFVHFGIAQSSTRGGSRVEGIFAGLFMNA
YGAGQVMLRWGVLAKSVKNIMLGHASVQAEMEQVVEVYEYAQKLGGEAGF
YHILNNPKASLLSLTQFPHFSSVVLGNAAGLGIMGEYRGTPRNQDLYDAA
KAYAEQLKENGVINYSVLDLTAEELEAIKHQLNPKDNDVEL*
RSV Gene Sequence SP9 Codon Optimized:
RSV F (SEQ ID NO. 5)
ATGGAGCTGCTCATCTTGAAGGCTAACGCCATTACCACTATCCTTACAGC
GGTGACGTTCTGCTTTGCATCCGGTCAGAATATTACCGAAGAGTTCTACC
AATCTACTTGTAGCGCTGTCTCAAAAGGCTATCTGTCGGCCCTCCGTACA
GGATGGTACACGAGTGTTATCACCATCGAATTGTCCAACATTAAGGAGAA
TAAATGCAACGGTACTGACGCGAAGGTAAAGCTTATCAAACAGGAACTGG
ATAAGTACAAGAACGCAGTGACAGAGCTCCAATTGCTGATGCAGTCTACC
CCCCCTACGAATAACCGCGCTAGGAGAGAACTTCCACGATTCATGAACTA
TACTCTCAATAACGCCAAAAAGACCAACGTCACATTGAGCAAAAAGCGTA
AGCGCAGGTTTCTGGGCTTCCTCCTGGGAGTTGGTTCAGCTATTGCGTCG
GGCGTAGCCGTGAGTAAAGTCCTTCACTTGGAGGGAGAAGTTAATAAGAT
CAAGTCCGCACTCCTGTCTACTAACAAAGCTGTGGTCAGCTTGTCAAACG
GTGTATCCGTGCTGACCTCGAAGGTTCTTGACCTCAAAAATTACATCGAT
AAGCAATTGCTGCCGATTGTCAACAAGCAGAGTTGTTCTATCAGCAATAT
TGAGACGGTGATCGAGTTCCAACAGAAAAACAACAGACTCCTGGAAATCA
CACGTGAGTTTTCAGTAAATGCCGGCGTTACTACCCCCGTCTCCACGTAC
ATGCTTACAAACTCGGAATTGCTCAGTCTGATTAACGACATGCCTATCAC
TAATGATCAGAAGAAGCTTATGTCTAACAACGTGCAAATTGTCCGCCAGC
AAAGCTATTCCATCATGTCAATCATTAAAGAGGAAGTGTTGGCGTACGTA
GTTCAGCTCCCACTGTACGGAGTCATCGACACCCCGTGCTGGAAGCTTCA
TACCTCGCCCTTGTGTACGACAAATACTAAAGAGGGTTCTAACATTTGCC
TCACCAGGACGGATCGAGGCTGGTATTGCGATAACGCTGGAAGTGTGAGC
TTCTTCCCTCAAGCAGAAACATGTAAGGTACAGTCCAATAGAGTTTTTTG
CGACACTATGAACTCACTGACCCTTCCATCTGAGATCAATTTGTGTAACG
TCGATATCTTCAACCCGAAGTACGACTGCAAAATTATGACGTCCAAGACA
GATGTGTCGAGTAGCGTAATCACTTCACTCGGTGCCATCGTTTCTTGCTA
CGGCAAGACCAAATGTACGGCTTCCAATAAGAACCGTGGAATTATCAAAA
CATTCTCGAACGGTTGCGACTATGTCAGCAATAAGGGCATGGACACTGTG
AGTGTAGGAAACACCCTGTACTACGTTAACAAGCAAGAAGGTAAATCACT
GTATGTCAAGGGCGAGCCCATTATCAATTTTTACGATCCTCTTGTGTTCC
CATCCGACGAGTTCGATGCGTCTATCAGCCAGGTAAACGAAAAGATTAAC
CAGTCCTTGGCATTTATCCGCAAATCGGACGAGCTCCTGCACAATGTTAA
CGCCGGAAAGAGTACGACAAACATTATGATCACTACCATCATTATCGTCA
TTATCGTGATCCTTTTGTCACTCATTGCTGTAGGTCTGCTTTTGTACTGT
AAAGCGAGGTCTACGCCCGTTACACTCAGCAAGGATCAACTGTCCGGCAT
CAATAACATTGCCTTCTCGAATTAA
RSV G (SEQ ID NO. 6)
ATGTCCAAGAACAAAGACCAGCGTACCGCTAAGACTCTGGAGCGCACATG
GGATACGCTCAATCACTTGCTTTTCATCTCTAGCTGCCTGTACAAACTCA
ACTTGAAGTCAGTGGCCCAAATTACCCTTTCGATCCTGGCGATGATTATC
AGTACTTCCCTCATCATTGCAGCTATCATTTTTATCGCCTCTGCGAATCA
TAAGGTCACACCCACGACCGCAATCATTCAGGACGCTACTAGCCAAATCA
AAAACACAACCCCTACGTATTTGACTCAGAACCCACAACTGGGTATTTCA
CCGTCGAATCCCAGTGAAATCACCTCCCAGATCACAACTATTCTTGCCTC
TACCACGCCTGGCGTTAAGAGCACACTCCAATCAACTACCGTAAAGACGA
AAAACACAACTACCACCCAGACGCAGCCATCCAAGCCGACAACTAAACAA
AGGCAGAACAAGCCCCCTTCGAAGCCAAATAACGATTTCCACTTCGAGGT
GTTTAACTTCGTCCCGTGTAGTATCTGCTCTAATAACCCCACCTGTTGGG
CTATTTGCAAAAGAATCCCTAACAAGAAGCCAGGAAAAAAGACGACAACT
AAACCCACCAAGAAGCCTACGTTGAAAACAACTAAGAAGGACCCGAAACC
ACAAACCACGAAGAGCAAAGAAGTTCCCACAACTAAGCCTACCGAGGAAC
CGACGATCAATACAACTAAGACCAACATTATCACGACACTGCTCACTTCA
AATACCACTGGTAACCCAGAGCTGACCTCCCAGATGGAAACCTTCCATTC
GACGAGTTCTGAGGGCAACCCCAGCCCTTCCCAAGTATCAACAACTTCGG
AATACCCATCTCAGCCCAGTAGCCCTCCGAATACCCCACGACAATAA
RSV M (SEQ ID NO. 7)
ATGGAGACCTACGTGAACAAGCTGCACGAAGGTTCCACTTATACAGCTGC
CGTCCAGTACAATGTTCTCGAGAAAGACGATGACCCCGCGTCTTTGACGA
TCTGGGTTCCTATGTTCCAAAGCTCAATGCCAGCAGATCTTCTGATTAAG
GAACTCGCTAACGTGAATATCTTGGTCAAACAGATTTCGACCCCGAAGGG
CCCCTCGCTTCGTGTTATGATCAACTCCCGCTCTGCCGTACTGGCGCAAA
TGCCTAGCAAGTTTACTATCTGCGCAAACGTGTCACTCGACGAGAGGTCG
AAATTGGCTTACGATGTCACAACGCCATGTGAAATTAAGGCCTGCAGTCT
GACCTGTCTTAAGTCCAAAAATATGCTCACTACAGTTAAGGACTTGACCA
TGAAAACGCTGAACCCGACTCATGACATCATTGCTCTCTGCGAGTTCGAA
AACATCGTGACCTCTAAGAAGGTCATCATTCCCACATATCTGAGAAGCAT
CTCAGTACGAAATAAAGATCTTAACACTTTGGAGAACATTACCACGACAG
AATTTAAGAATGCGATCACTAACGCCAAGATCATTCCTTACTCCGGACTC
CTGTTGGTGATCACCGTTACGGACAACAAAGGTGCATTTAAGTACATTAA
ACCACAGTCGCAATTCATCGTCGATCTGGGCGCTTATCTTGAGAAGGAGA
GTATCTACTACGTGACAACTAATTGGAAGCACACCGCCACCCGTTTCGCG
ATTAAACCGATGGAAGATTAA
RSV N (SEQ ID NO. 8)
GGTACCGGATCCGCCACCATGGCTCTGTCCAAGGTCAAGCTGAACGACAC
CCTGAACAAGGACCAGCTGCTGTCCTCCTCCAAGTACACCATCCAGCGTT
CCACCGGTGACTCCATCGACACCCCCAACTACGACGTGCAGAAGCACATC
AACAAGCTGTGCGGCATGCTGCTGATCACCGAGGACGCTAACCACAAGTT
CACCGGTCTGATCGGCATGCTGTACGCTATGTCCCGTCTGGGTCGTGAGG
ACACCATCAAGATCCTGCGTGACGCTGGTTACCACGTGAAGGCTAACGGT
GTCGACGTGACCACCCACCGTCAGGACATCAACGGCAAGGAGATGAAGTT
CGAGGTCCTGACCCTGGCTTCCCTGACCACCGAGATCCAGATCAACATCG
AGATCGAGTCCCGTAAGTCCTACAAGAAGATGCTGAAGGAGATGGGCGAG
GTCGCCCCCGAGTACCGTCACGACTCCCCCGACTGCGGCATGATCATCCT
GTGCATCGCTGCTCTCGTCATCACCAAGCTGGCTGCTGGTGACCGTTCCG
GTCTGACCGCTGTGATCCGTCGTGCTAACAACGTGCTGAAGAACGAGATG
AAGCGCTACAAGGGTCTGCTGCCCAAGGACATCGCTAACAGCTTCTACGA
GGTGTTCGAGAAGCACCCCCACTTCATCGACGTGTTCGTGCACTTCGGTA
TCGCTCAGTCCTCCACCCGTGGTGGTTCCCGTGTGGAGGGCATCTTCGCT
GGTCTGTTCATGAACGCTTACGGTGCTGGCCAGGTCATGCTGCGTTGGGG
TGTGCTGGCTAAGTCCGTGAAGAACATCATGCTGGGTCACGCTTCCGTGC
AGGCTGAGATGGAGCAGGTGGTGGAGGTGTACGAGTACGCTCAGAAGCTG
GGCGGCGAGGCTGGTTTCTACCACATCCTGAACAACCCCAAGGCTTCCCT
GCTGTCCCTGACCCAGTTCCCCCACTTCTCCTCCGTGGTGCTGGGTAACG
CTGCTGGTCTGGGTATCATGGGCGAGTACCGTGGCACCCCCCGTAACCAG
GACCTGTACGACGCTGCTAAGGCTTACGCCGAGCAGCTCAAGGAGAACGG
CGTCATCAACTACTCCGTGCTGGACCTGACCGCTGAGGAGCTGGAGGCTA
TCAAGCACCAGCTGAACCCCAAGGACAACGACGTGGAGCTGTAATAAAAG
CTT

### Example 2

### Transfection of SF9 Insect Cells to Make Recombinant Virus Stocks and Plaque Purification

Different bacmid DNA from above were picked for each construct and were isolated. These DNAs were precipitation and added to SF9 cells for 5 hours. Cells were counted at harvest (68-74 hours post bacmid addition). Each transfection (3 transfections/construct) comprised 10⁻¹ to 10⁻⁷ cells. The cells were plated and overlayed. The cells were incubated for 7 to 11 days. Next, 10 to 12 plaques from each construct were selected and isolated. The plaque plugs were transferred to 1 ml media and eluted overnight.

### Example 3

### Infecting Insect Cells with Primary Virus Stock

Next, 30 ml of insect cells (2×10⁶ cells/ml) were infected with 0.3 ml of plaque eluate and incubated 68-72 hours. Cultures for each construct, preferably from 3 different transfections, were started. The cells were counted at harvest (68-74 hours post infection). Approximately 1 ml of culture for expression analysis was centrifuged and the pellet and supernatant were saved for testing.

### Example 4

### Protein Expression Analysis

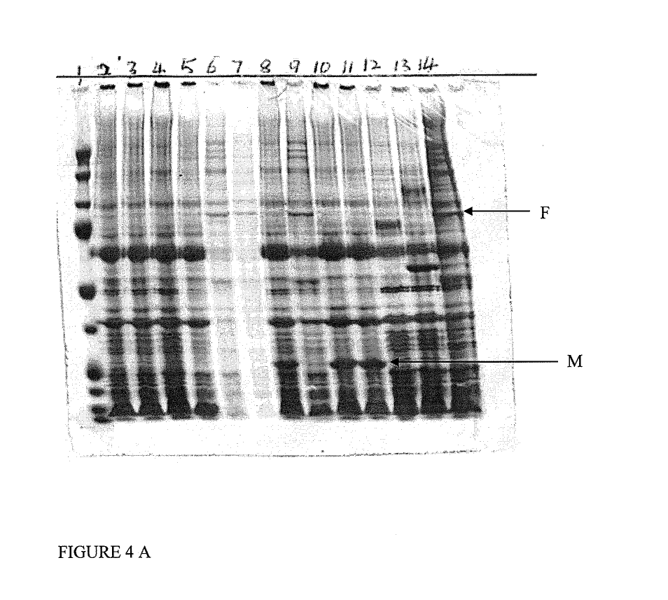

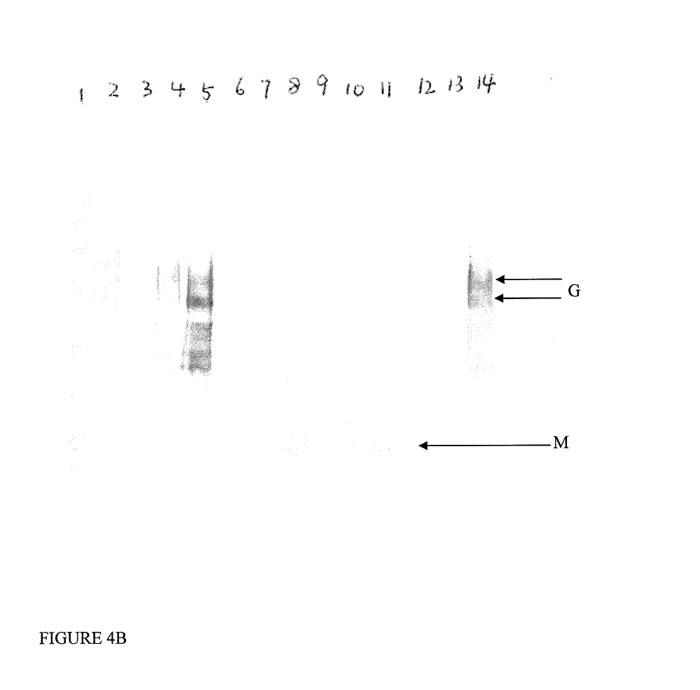

Protein expression analysis was done on the cell pellet and on the supernatant of infected cells. The cell pellet was re-suspended in 1 ml PBS (equal volume to the culture sample) and stored at −20° C. Equal volumes of cell samples and 2× sample buffer containing βME (beta-mercaptoehtanol) were loaded, approximately 15 to 20 μl (about to 7.5 to 10 μl of the culture)/lane, onto SDS Laemmli gels (one gel for staining, the other gel for a western blot, sometimes more than 1 blot depending on construct and antibodies used). A similar process was used for supernatant analysis. The constructs were loaded as follows: RSV F, RSV G and Influenza M1 co-expression (lane 2), RSV F and Influenza M1 (lane 3) co-expression, RSV G and Influenza M1 (lane 4) co-expression, RSV G (lane 5), RSV F (lanes 6 and 7), RSV F and RSV M (lanes 8 and 9), RSV M (lanes 10 and 11), A/H3N2/Fujian/HANAM1 control (lane 12), HIV VLP control (lane 13), and RSV F, RSV M and RSV G co-infection (lane 14) (FIG. 4).

For VLP protein expression analysis, a culture of insect cells was infected at ˜3 MOI (Multiplicity of infection=virus ffu or pfu/cell). The culture and supernatant were harvested 48-72 post-infection. Next, the supernatant was centrifuged at 100,000×g for 1 hour, and the supernatant removed and saved. The pellet was resuspended in PBS at 0.02 to 0.01 of original culture volume pelleted. The analysis was the same as described above.

### Example 5

### Expression of RSV M Protein Alone Forms VLPs

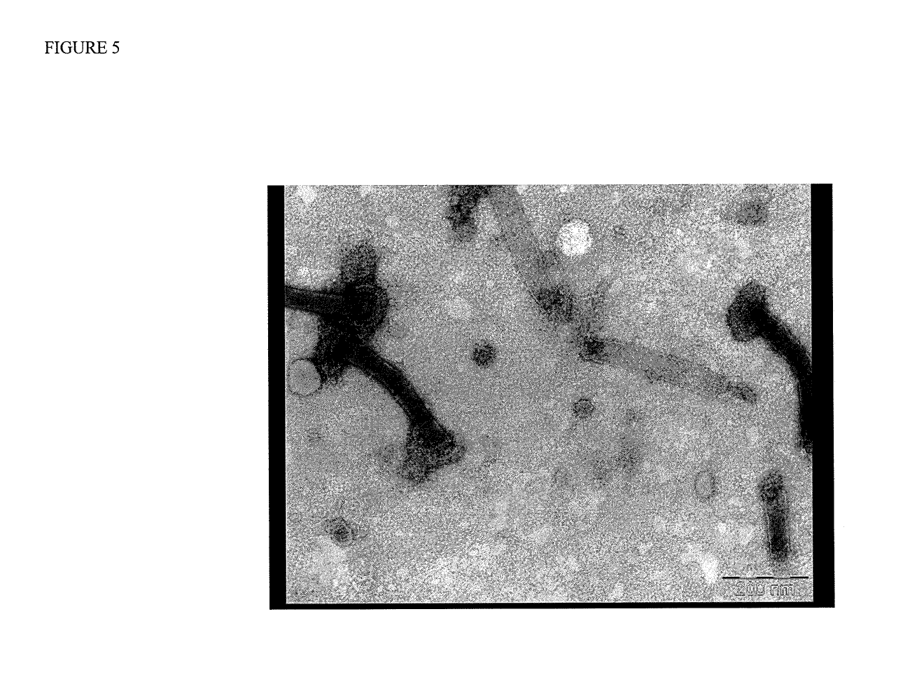

A construct comprising only RSV M was expressed in SF9 cells and analyzed according the above procedures. A SDS gel and a western blot of the isolated supernatant are shown on FIG. 4. As shown in lanes 10 and 11, expression of RSV M protein alone lead to the formation of RSV-VLPs. The gel and blot has the expected band at a molecular weight of about 28 k and is recognized by RSV antiserum. Thus, RSV alone is sufficient to form a core virus like particle. FIG. 5 represents an electron micrograph of RSV VLPs with ammonium molybdate staining the rod shaped particles are baculovirus and round particles are RSV-VLPs.

### Example 6

### RSV VLPs Comprising RSV G Protein

A construct comprising RSV M and RSV G was expressed in SF9 cells and analyzed according the above procedures. A SDS gel and a western blot of the supernatant are shown on FIG. 4. Co-expression of RSV M and RSV G proteins leads to the formation of VLPs as shown on lane 14 in FIG. 4A and lane 5 to 8 in FIG. 4B.

### Example 7

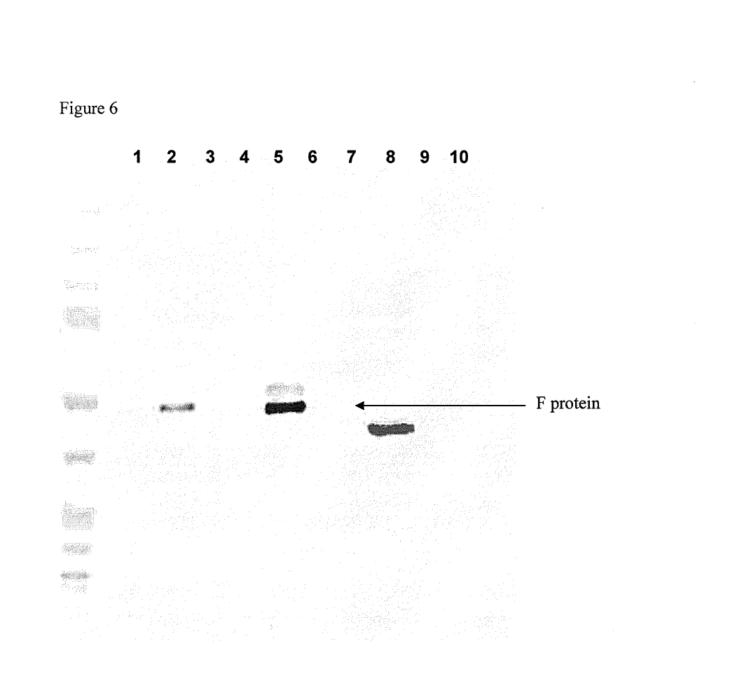

Constructs comprising RSV G (lane 1) RSV F (lane 2), RSV M (lane 3), RSV F and RSV M (lane 4), RSV NP (lane 5), RSV F1 with Flu H5N1 Indo HA C-term (lane 6), Avian Influenza A/H5N1 Indo HANA M1 control (lane 7), and Sf9 cell blank as negative control (lane 8) were infected and expressed in Sf9 cells (FIG. 6). Particles were purified from Sf9 cell culture expressing the above referenced constructs through the 30% sucrose cushion. In this system the VLPs in the culture media will pellet when the cushion with the media is centrifuged. Western blots (FIG. 6) were performed using the VLP pellet. The blot depicted in FIG. 6 was probed with a primary mouse antibody to RSV (ascetic fluid) that recognizes F0 and F1 (Covanlab, Cat. CVL-MAB0040) and secondary antibody goat anti-mouse IgG (KPL, Cat. 075-1806). As shown in lanes 4 (RSV F and M) and lane 5 (RSV F1 fused to the c-term end of flu H5N₁ HA) the antibodies reacted with protein on the blot. This indicates that VLPs are present and that VLPs that comprise RSV F were made. The band in lane 2 (RSV F only) may indicate that F protein is forming VLPs or, more probable, that F1 is forming a conglomeration that pellet like VLPs in the 30% sucrose cushion. However, co-expression of RSV M and F do result in a brighter band, which is indicative of not only VLP formation but more efficient VLP formation. Interestingly, the F1-fused to the C-term of influenza HA resulted in formation of VLPs. This result corresponds with data that the c-terminal portion of influenza HA is all that required to form VLPs (see co-pending application 60/940,201, filed May 25, 2007, herein incorporated by reference in its entirety). Thus, these data confirm that RSV VLPs are forming.

### Example 8

In these set of experiments RSV F proteins variants were made, cloned into baculorivs and expressed in Sf9 cells to determine if VLPs were formed. RSVF1-Indo and F1-SP are engineered genes. RSV F1-Indo has fusion domain deleted and no signal peptide. In addition, this gene has influenza HA transmembrane domain and C-terminus (derived from A/Indonesia/5/05 (H5N1) strain). The RSV F1-SP has the fusion domain deleted. In place of the fusion domain, this protein has the signal peptide from the F2. The F1-SP protein has wild type RSV F1 transmembrane domain and c-terminal sequence, unless indicated otherwise indicated. The following are the amino acid and nucleotide sequences of the above described constructs.

| | |
|---|---|
| RSV F1-Indo Chimeric Amino Acid Sequence | (SEQ ID NO 9) |

MEGEVNKIKS ALLSTNKAVV SLSNGVSVLT SKVLDLKNYI DKQLLPIVNK
QSCSISNIET VIEFQQKNNR LLEITREFSV NAGVTTPVST YMLTNSELLS
LINDMPITND QKKLMSNNVQ IVRQQSYSIM SIIKEEVLAY VVQLPLYGVI
DTPCWKLHTS PLCTTNTKEG SNICLTRTDR GWYCDNAGSV SFFPQAETCK
VQSNRVFCDT MNSLTLPSEI NLCNVDIFNP KYDCKIMTSK TDVSSSVITS
LGAIVSCYGK TKCTASNKNR GIIKTFSNGC DYVSNKGMDT VSVGNTLYYV
NKQEGKSLYV KGEPIINFYD PLVFPSDEFD ASISQVNEKI NQSLAFIRKS
DELLHNVNAG KSTTNIMQIL SIYSTVASSL ALAIMMAGLS LWMCSNGSLQ
CRICI

| | |
|---|---|
| RSV F1-Indo Chimeric Nucleotide Sequence | (SEQ ID NO 10) |

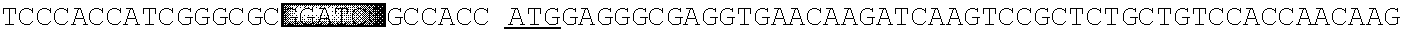

GCTGTGGTGTCCCTGTCCAACGGTGTCTCCGTGCTGACCTCCAAGGTGCTGGACCTGAAGAACTACATCGACAAGCAGC
TGCTGCCCATCGTGAACAAGCAGTCCTGCTCCATCTCCAACATCGAGACCGTGATCGAGTTCCAGCAGAAGAACAACCG
TCTGCTCGAGATCACCCGTGAGTTCTCCGTGAACGCTGGTGTCACCACCCCCGTGTCCACCTACATGCTGACCAACTCC
GAGCTGCTGTCCCTGATCAACGACATGCCCATCACCAACGACCAAAAGAAGCTGATGTCCAACAACGTGCAGATCGTGC
GTCAGCAGTCCTACTCTATCATGTCCATCATCAAGGAAGAAGTCCTGGCTTACGTCGTGCAGCTGCCCCTGTACGGTGT
CATCGACACCCCCTGCTGGAAGCTGCACACCTCCCCCCTGTGCACCACCAACACCAAGGAAGGTTCCAACATCTGCCTG
ACCCGTACCGACCGTGGTTGGTACTGCGACAACGCTGGTTCCGTGTCCTTCTTCCCCCAAGCTGAGACCTGCAAGGTGC
AGTCCAACCGTGTGTTCTGCGACACCATGAACTCCCTGACCCTGCCCTCCGAGATCAACCTGTGCAACGTGGACATCTT
CAACCCCAAGTACGACTGCAAGATCATGACCTCTAAGACCGACGTGTCCTCCTCCGTGATCACCTCCCTGGGTGCTATC
GTGTCCTGCTACGGCAAGACCAAGTGCACCGCTTCCAACAAGAACCGCGGCATCATCAAGACCTTCTCCAACGGTTGCG
ACTACGTGTCCAACAAGGGCATGGACACCGTGTCCGTCGGTAACACCCTGTACTACGTCAACAAGCAGGAAGGCAAGTC
TCTGTACGTGAAGGGCGAGCCCATCATCAACTTCTACGACCCCCTGGTGTTCCCCTCCGACGAGTTCGACGCTTCCATC
AGCCAGGTCAACGAGAAGATCAACCAGTCCCTGGCTTTCATCCGTAAGTCCGACGAGCTGCTGCACAACGTCAACGCTG
GCAAGTCTACCACCAACATCATGCAGATCCTGTCCATCTACTCCACCGTGGCTTCCTCCCTGGCTCTGGCTATCATGAT

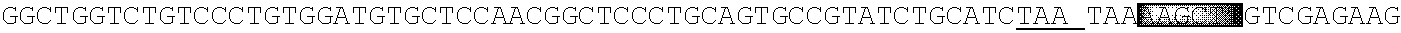

TACTAGAGGATCAT

| | |
|---|---|
| RSV F1-SP (with signal peptide)Amino Acid Sequence | (SEQ ID NO 11) |

MELLILKANA ITTILTAVTF CFASGQNSKV LHLEGEVNKI KSALLSTNKA
VVSLSNGVSV LTSKVLDLKN YIDKQLLPIV NKQSCSISNI ETVIEFQQKN
NRLLEITREF SVNAGVTTPV STYMLTNSEL LSLINDMPIT NDQKKLMSNN
VQIVRQQSYS IMSIIKEEVL AYVVQLPLYG VIDTPCWKLH TSPLCTTNTK
EGSNICLTRT DRGWYCDNAG SVSFFPQAET CKVQSNRVFC DTMNSLTLPS
EINLCNVDIF NPKYDCKIMT SKTDVSSSVI TSLGAIVSCY GKTKCTASNK
NRGIIKTFSN GCDYVSNKGM DTVSVGNTLY YVNKQEGKSL YVKGEPIINF
YDPLVFPSDE FDASISQVNE KINQSLAFIR KSDELLHNVN AGKSTTNIMI
TTIIIVIIVI LLSLIAVGLL LYCKARSTPV TLSKDQLSGI NNIAFSN

| | |
|---|---|
| RSV F1-SP (with signal peptide)Nucleotide Sequence | (SEQ ID NO 12) |

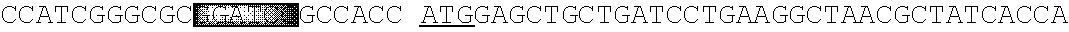

CCATCCTGACCGCTGTGACCTTCTGCTTCGCTTCCGGCCAGAACTCCAAGGTGCTGCACC
TCGAGGGCGAGGTGAACAAGATCAAGTCCGCTCTGCTGTCCACCAACAAGGCTGTGGTGT
CCCTGTCCAACGGTGTCTCCGTGCTGACCTCCAAGGTCCTGGACCTGAAGAACTACATCG
ACAAGCAGCTGCTGCCCATCGTGAACAAGCAGTCCTGCTCCATCTCCAACATCGAGACCG
TGATCGAGTTCCAGCAGAAGAACAACCGTCTGCTCGAGATCACCCGTGAGTTCTCCGTGA
ACGCTGGTGTCACCACCCCCGTGTCCACCTACATGCTGACCAACTCCGAGCTGCTGTCCC
TGATCAACGACATGCCCATCACCAACGACCAGAAAAAGCTGATGTCCAACAACGTGCAGA
TCGTGCGTCAGCAGTCCTACTCTATCATGTCCATCATCAAGGAAGAGGTCCTGGCTTACG
TGGTGCAGCTGCCCCTGTACGGTGTCATCGACACCCCCTGCTGGAAGCTGCACACCTCCC
CCCTGTGCACCACCAACACCAAGGAAGGTTCCAACATCTGCCTGACCCGTACCGACCGTG
GTTGGTACTGCGACAACGCTGGTTCCGTGTCCTTCTTCCCCCAAGCTGAGACCTGCAAGG
TGCAGTCCAACCGTGTGTTCTGCGACACCATGAACTCCCTGACCCTGCCCTCCGAGATCA
ACCTGTGCAACGTGGACATCTTCAACCCCAAGTACGACTGCAAGATCATGACCTCTAAGA
CCGACGTGTCCTCCTCCGTGATCACCTCCCTGGGTGCTATCGTGTCCTGCTACGGCAAGA
CCAAGTGCACCGCTTCCAACAAGAACCGCGGCATCATCAAGACCTTCTCCAACGGTTGCG
ACTACGTGTCCAACAAGGGCATGGACACCGTGTCCGTCGGTAACACCCTGTACTACGTCA
ACAAGCAGGAAGGCAAGTCTCTGTACGTGAAGGGCGAGCCCATCATCAACTTCTACGACC
CCCTGGTGTTCCCCTCCGACGAGTTCGACGCTTCCATCAGCCAGGTCAACGAGAAGATCA
ACCAGTCCCTGGCTTTCATCCGTAAGTCCGACGAGCTGCTGCACAACGTCAACGCTGGCA
AGTCTACCACCAACATCATGATCACCACTATCATCATCGTGATCATCGTCATCCTGCTGT
CTCTCATCGCTGTGGGTCTGCTGCTGTACTGCAAGGCTCGTTCCACCCCTGTGACCCTGT

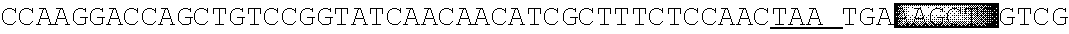

| | |
|---|---|
| A/Indonesia/5/05 M1 | (SEQ ID NO 13) |

ATGAGTCTTCTAACCGAGGTCGAAACGTACGTTCTCTCTATCATCCCGTCAGGCCCCCTCAAAGCCGAGATCGCGCAGAAACT
TGAAGATGTCTTTGCAGGAAAGAACACCGATCTCGAGGCTCTCATGGAGTGGCTGAAGACAAGACCAATCCTGTCACCTCTGA
CTAAAGGGATTTTGGGATTTGTATTCACGCTCACCGTGCCCAGTGAGCGAGGACTGCAGCGTAGACGCTTTGTCCAGAATGCC
CTAAATGGAAATGGAGATCCAAATAATATGGATAGGGCAGTTAAGCTATATAAGAAGCTGAAAAGAGAAATAACATTCCATGG
GGCTAAAGAGGTTTCACTCAGCTACTCAACCGGTGCACTTGCCAGTTGCATGGGTCTCATATACAACAGGATGGGAACGGTGA
CTACGGAAGTGGCTTTTGGCCTAGTGTGTGCCACTTGTGAGCAGATTGCAGATTCACAGCATCGGTCTCACAGGCAGATGGCA
ACTATCACCAACCCACTAATCAGGCATGAAAACAGAATGGTGCTGGCCAGCACTACAGCTAAGGCTATGGAGCAGATGGCGGG
ATCAAGTGAGCAGGCAGCGGAAGCCATGGAGGTCGCTAATCAGGCTAGGCAGATGGTGCAGGCAATGAGGACAATTGGAACTC
ATCCTAACTCTAGTGCTGGTCTGAGAGATAATCTTCTTGAAAATTTGCAGGCCTACCAGAAACGAATGGGAGTGCAGATGCAG
CGATTCAAGTGA

| | |
|---|---|
| Optimized Synthetic Influenza A/Fujian/411/02 H3N2 M1 | (SEQ ID NO 14) |

TTCATACCGTCCCACCATCGGGCGCGGATCCGCCACC ATGTCCCTGCTGACCGAGGTGGAGACCTACGTGCTGTCCATC
GTGCCCTCCGGTCCTCTGAAGGCTGAGATCGCTCAGAGGCTCGAGGACGTGTTCGCTGGCAAGAACACCGACCTCGAGG
CTCTGATGGAGTGGCTCAAGACCCGTCCCATCCTGTCCCCCCTGACCAAGGGTATCCTGGGTTTCGTGTTCACCCTGAC
CGTGCCTTCCGAGCGTGGTCTGCAGCGTCGTCGTTTCGTGCAGAACGCTCTGAACGGTAACGGTGACCCCAACAACATG
GACAAGGCCGTGAAGCTGTACCGTAAGCTGAAGCGTGAGATCACCTTCCACGGTGCTAAGGAGATCGCTCTGTCCTACT
CCGCTGGTGCTCTGGCTTCCTGCATGGGCCTGATCTACAACCGTATGGGTGCTGTGACCACCGAGGTGGCCTTCGGTCT
GGTCTGCGCTACCTGCGAGCAGATCGCTGACTCCCAGCACCGTTCCCACCGTCAGATGGTGGCTACCACCAACCCCCTG
ATCCGTCACGAGAACCGCATGGTGCTGGCTTCCACCACCGCTAAGGCTATGGAGCAGATGGCTGGTTCCTCCGAGCAGG
CTGCTGAGGCCATGGAGATCGCTTCCCAGGCTCGCCAGATGGTGCAGGCTATGCGTGCTATCGGCACCCACCCCTCCTC
CTCCACCGGTCTGCGTGACGACCTGCTCGAGAACCTGCAGACCTACCAAAAGCGTATGGGTGTGCAGATGCAGCGCTTC
AAGTAA TAAAAGCTtGTCGAGAAGTACTAGa

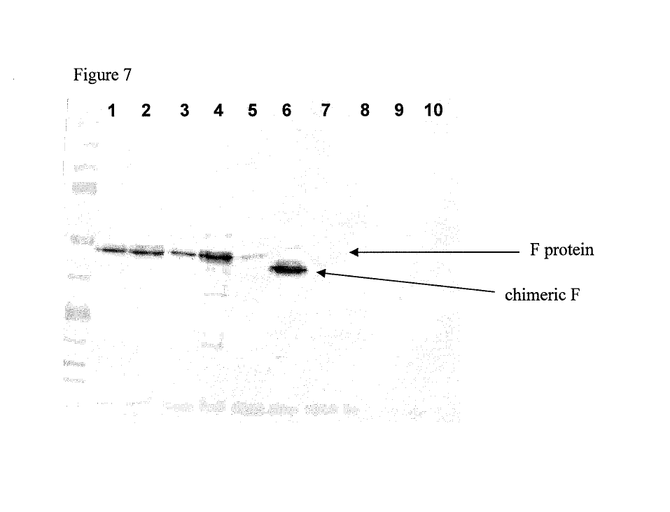

Constructs comprising RSV F1/SP (lane 1), RSV F1/SP and RSV M co-infection (lane 2), RSV F1/SP and RSV M and RSV G co-infection (lane 3), RSV F1/SP with Indo M1 co-infection (lane 4), RSV F1/SP and Indo M1 and RSV G co-infection (lane 5), RSV F1/H5N1 Indo TM-CT of HA (lane 6), RSV M (lane 7), Avian Influenza A/H5N₁ WT baculovirus VLP pellets as control (lane 8), WCB101 Sf9 cell blank as negative control (lane 9) were infected and expressed in Sf9 cells (FIG. 7). Particles were purified from Sf9 cells infected with the above referenced constructs through a 30% sucrose cushion. A western blot (FIG. 7) was performed using the VLP pellet. The blot was probed with a primary mouse antibody to RSV (ascetic fluid) that recognizes F0 and F1 (Covanlab, Cat. CVL-MAB0040) and secondary antibody goat anti-mouse IgG (KPL, Cat. 075-1806). As shown in lanes 1, 2, and 3, cells expressing RSV F alone and with RSV M have F protein on the blots. The data also show that RSV F co-expressed with influenza M1 also formed VLPs (lane 4). However, when comparing lanes 1, 2 and 3 with lane number 4 there is as a significant increase in the amount of F1 seen in the pellet. This indicates that chimeric F protein (when fused to influenza TM-CT of HA) when co-expressed with influenza M1, not only produces VLPs that express F protein, but is more efficient. In addition, when chimeric F protein is expressed alone, VLPs are formed. Again, this result corresponds with data that only the c-terminal portion of influenza HA is all that is needed to form VLPs (see co-pending application 60/940,201, filed May 25, 2007, herein incorporated by reference in its entirety). Thus, these also data confirms that RSV VLPs are forming.

All publications, patents and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A virus like particle (VLP) comprising a respiratory syncytial virus (RSV) M protein.

2. The virus like particle of claim 1, wherein said VLPs comprises additional RSV proteins.

3. The VLP of claim 2, wherein said VLP comprises a RSV F protein.

4. The VLP of claim 2, wherein said VLP comprises RSV G protein.

5. The VLP of claim 4, wherein the G protein is from RSV group A.

6. The VLP of claim 4, wherein the G protein is from RSV group B.

7. The VLP of claim 1, wherein said VLP comprises a chimeric molecule.

8. The VLP of claim 7, wherein said chimeric molecule comprises a RSV protein, or portion thereof, that associates with RSV M.

9. The VLP of claim 8, wherein said chimeric molecule comprises the transmembrane domain and/or cytoplasmic tail of RSV G protein.

10. The VLP of claim 9, wherein said chimeric molecule comprises the cytoplasmic and/or the transmembrane domain of RSV G protein fused to the HA and/or NA protein, or portion thereof, of influenza virus.

11. The VLPs of claim 10, wherein said HA and/or NA transmembrane and/or cytoplasmic domain has been replaced with said transmembrane and/or cytoplasmic domain from RSV G protein.

12. The VLP of claim 3, wherein said VLP comprises RSV N.

13. The VLP of claim 4, wherein said VLP comprises RSV N.

14. The VLP of claim 1, wherein said VLP is expressed in a eukaryotic cell under conditions which permit the formation of VLPs.

15. The VLP of claim 14, wherein said eukaryotic cell is selected from the group consisting of yeast, insect, amphibian, avian or mammalian cells.

16. A VLP comprising a chimeric F protein from RSV and a M protein from an influenza virus, wherein said chimeric F protein comprises a F protein fused to the transmembrane domain and cytoplasmic tail of an influenza glycoprotein.

17. The VLP of claim 9, wherein said influenza glycoprotein is HA.

18. The VLP of claim 9, wherein said influenza glycoprotein is NA.

19. The chimeric F protein, wherein said F protein transmembrane and cytoplasmic domains are replaced with influenza HA and/or NA protein transmembrane and cytoplasmic domains.

20. A method of producing a VLP, comprising transfecting vectors encoding at least one RSV protein into a suitable host cell and expressing said RSV virus protein under conditions that allow VLP formation.

21. The method of claim 20, wherein said VLP comprises a F and/or M protein from RSV.

22. The method of claim 21, wherein said VLP comprises the G protein from RSV group A.

23. The method of claim 21, wherein said VLP comprises the G protein from RSV group B.

24. The method of claim 20, wherein said F protein is a chimeric F protein wherein said chimeric F protein comprises an F protein fused to the transmembrane domain and cytoplasmic tail of an influenza glycoprotein.

25. The VLP of claim 24, wherein said influenza glycoprotein is HA.

26. The VLP of claim 24, wherein said influenza glycoprotein is NA.

27. The method of claim 24, wherein said VLP further comprises a M protein derived from an influenza virus.

28. The method of claim 27, wherein said VLP comprises the G protein from RSV group A.

29. The method of claim 27, wherein said VLP comprises the G protein from RSV group B.

30. The method of claim 20, wherein said eukaryotic cell is selected from the group consisting of yeast, insect, amphibian, avian or mammalian cells.

31. An antigenic formulation comprising a VLP which comprises at least one RSV protein.

32. The antigenic formulation of claim 31, wherein said VLP comprises a RSV M protein.

33. The antigenic formulation of claim 31, wherein said VLP comprises a RSV F protein.

34. The antigenic formulation of claim 33, wherein said VLP further comprises a RSV G protein.

35. The antigenic formulation of claim 34, wherein said VLP comprises the G protein from RSV group A.

36. The antigenic formulation of claim 34, wherein said VLP comprises the G protein from RSV group B.

37. An antigenic formulation comprising a VLP which comprises a chimeric F protein from a RSV and M protein derived from an influenza virus, wherein said chimeric F protein is a fused to the transmembrane domain and cytoplasmic tail of influenza HA protein.

38. The antigenic formulation of claim 31, further comprising an adjuvant.

39. The antigenic formulation of claim 38, wherein said adjuvant are Novasomes.

40. The antigenic formulation of claims 31, wherein said formulation is suitable for human administration.

41. The antigenic formulation of claim 31, wherein different antigenic RSV VLPs are blended together to create a multivalent formulation.

42. The antigenic formulation claim 31, wherein the formulation is administered to the subject orally, intradermally, intranasally, intramuscularly, intraperitoneally, intravenously, or subcutaneously.

43. A vaccine comprising a VLP which comprises at least one RSV protein.

44. The vaccine of claim 43, wherein said VLP comprises a RSV M protein.

45. The vaccine of claim 43, wherein said VLP comprises a RSV F protein.

46. The vaccine of claim 45, wherein said VLP comprises a RSV G protein.

47. The vaccine of claim 46, wherein said G protein is from RSV group A.

48. The vaccine of claim 46, wherein said G protein is from RSV group B.

49. A vaccine comprising a VLP which comprises a chimeric F protein from a RSV and a M protein from an influenza virus, wherein said chimeric F protein comprises an F protein fused to the transmembrane domain and cytoplasmic tail of an influenza glycoprotein.

50. The VLP of claim 49, wherein said influenza glycoprotein is HA.

51. The VLP of claim 49, wherein said influenza glycoprotein is NA.

52. The vaccine of claim 43, further comprising an adjuvant.

53. The vaccine of claim 52, wherein said adjuvant are Novasomes.

54. The vaccine of claim 43, wherein different antigenic RSV VLPs are blended together to create a multivalent formulation.

55. The vaccine claim 43, wherein the formulation is administered to a mammal orally, intradermally, intranasally, intramuscularly, intraperitoneally, intravenously, or subcutaneously.

56. A method of vaccinating a mammal against RSV comprising administering to said mammal a protection-inducing amount of a VLP comprising at least one RSV protein.

57. The method of claim 56, wherein said VLP comprises RSV M protein.

58. The method of claim 56, wherein said VLP comprises a RSV F protein.

59. The method of claim 58, wherein said VLP comprises the G protein from RSV group A.

60. The method of claim 58, wherein said VLP comprises the G protein from RSV group B.

61. The method of claim 56, wherein said VLP comprises a chimeric F protein from RSV and a M protein derived from an influenza virus, wherein said chimeric F protein is a fused to the transmembrane domain and cytoplasmic tail of influenza HA protein.

62. A method of inducing immunity to RSV infection or at least one symptom thereof in a subject, comprising administering at least one effective dose of RSV VLP.

63. The method of claim 62, wherein said VLP comprise RSV F

64. The method of claim 63, wherein said VLP comprises RSV M protein.

65. The method of claim 64, wherein said VLP further comprises a RSV G protein.

66. The method of claim 65, wherein said VLP comprises the G protein from RSV group A or group B.

67. The method of claim 62, wherein said VLP comprises a chimeric F protein from a RSV and a M protein derived from an influenza virus, wherein said chimeric F protein is a fused to the transmembrane domain and cytoplasmic tail of influenza HA protein.

68. The method of claim 62, wherein said subject is a mammal.

69. The method of claim 68, wherein said mammal is a human.

70. The method of claim 56, wherein said influenza VLP is formulated with an adjuvant or immune stimulator.

71. A chimeric virus like particle comprising a viral M from RSV and at least one protein from an infectious agent.

72. The VLP of claim 71, wherein said protein from an infectious agent is a viral protein.

73. The VLP of claim 71, wherein said protein from an infectious agent is an envelope associated protein.

74. The VLP of claim 71, wherein said protein from an infectious agent is expressed on the surface of the VLP.

75. The VLP of claim 72, wherein said protein from an infectious agent comprises an epitope that will generate a protective immune response in a vertebrate.

76. The VLP of claim 71, wherein said protein from another infectious agent can associated with RSV M protein.

77. The VLP of claim 71, wherein, said protein from another infectious agent is fused to a RSV protein.

78. The VLP of claim 77, wherein only a portion of said protein from another infectious agent is fused to said RSV protein.

79. The VLP of claim 78, wherein only a portion of said protein from another infectious agent is fused to a portion of said RSV protein.

80. The VLP of claim 78, wherein said portion of the protein from another infectious agent fused to said RSV protein is expressed on the surface said VLP.

81. The VLP of claim 77, wherein said RSV protein, or portion thereof, fused to the protein from another infectious agent associates with the RSV M protein.

82. The VLP of claim 77, wherein said RSV protein, or portion thereof, is from the group consisting of RSV F, G, N and P.

83. The VLPs of claim 71, wherein said VLP further comprises N and/or P protein from RSV.

84. The VLP of claim 71, wherein said VLPs comprise more than one protein from an infectious agent.

85. The VLP of claim 71, wherein said VLP comprise more one infectious agent protein.

86. The VLP of claim 72, wherein said viral protein is selected from a virus from the group consisting of influenza virus, dengue virus, yellow fever virus, herpes simplex virus I and II, rabies virus, parainfluenza virus, varicella zoster virus, human immunodeficiency virus, corona virus, West Nile virus and hepatitis virus.
